(19) United States
(12) Patent Application Publication
(10) Pub. No.: US 2024/0252381 A1
(43) Pub. Date: Aug. 1, 2024

(54) METHOD OF CORRECTING WALKING POSTURE OF USER AND WEARABLE DEVICE PERFORMING THE METHOD

(71) Applicant: Samsung Electronics Co., Ltd., Suwon-si (KR)

(72) Inventor: Daehyun KIM, Suwon-si (KR)

(21) Appl. No.: 18/635,957

(22) Filed: Apr. 15, 2024

Related U.S. Application Data
(63) Continuation of application No. PCT/KR2023/018340, filed on Nov. 15, 2023.

(30) Foreign Application Priority Data
Nov. 16, 2022 (KR) 10-2022-0153569
Jan. 11, 2023 (KR) 10-2023-0003959
Jul. 7, 2023 (KR) 10-2023-0088403

(51) Int. Cl. A61H 3/00; A63B 24/00

(52) U.S. Cl. CPC A61H 3/00; A63B 24/0006; A63B 24/0062; A63B 24/0087; A61H 2003/007; A63B 2220/16; A63B 2220/836; A63B 2225/20; A63B 2225/50; A63B 2230/625

(57) ABSTRACT

A method of controlling a wearable device may include: determining whether a walking state of a user of the wearable device is a normal state based on test movement information of the user obtained through test walking; when the walking state is not the normal state, determining first correction torque information based on the test movement information; and outputting a first correction torque corresponding to the first correction torque information through at least one of a drive module or an additional drive module.

### CROSS-REFERENCE TO RELATED APPLICATIONS

This application is continuation of International Application No. PCT/KR2023/018340 designating the United States, filed on Nov. 15, 2023, in the Korean Intellectual Property Receiving Office and claiming priority to Korean Patent Application No. 10-2022-0153569 filed on Nov. 16, 2022, Korean Patent Application No. 10-2023-0003959 filed on Jan. 11, 2023, and Korean Patent Application No. 10-2023-0088403 filed on Jul. 7, 2023, in the Korean Intellectual Property Office, the disclosures of which are all hereby incorporated by reference herein in their entireties.

### BACKGROUND

### 1. Field

Example embodiments relate to a technology for controlling a wearable device.

### 2. Description of Related Art

Aging demographics have contributed to a growing number of people who experience inconvenience and/or pain from reduced muscular strength or aging-induced joint problems. Thus, there is a growing interest in walking assist devices that enable elderly users or patients with reduced muscular strength or joint problems to walk with less effort and/or exercise.

### SUMMARY

According to an example embodiment, a wearable device may include: a base body configured to be disposed proximate to a waist portion of a user when the wearable device is worn on a body of the user; a waist support frame and a leg support frame configured to support at least a portion of the body of the user; a thigh fastener configured to attach the leg support frame to a thigh of the user; an inertial measurement unit (IMU) disposed within the base body; a drive module (comprising a motor and/or circuitry) configured to generate a torque to be applied to a leg of the user, the drive module being disposed between the waist support frame and the leg support frame; an angle sensor configured to measure a rotation angle of the leg support frame; and a control module including at least one processor configured to control the wearable device, wherein the leg support frame may include: a first partial leg support frame configured to be connected, directly or indirectly, to the drive module; a second partial leg support frame configured to be connected, directly or indirectly, to the thigh fastener; a hinge configured to connect, directly or indirectly, the first partial leg support frame and the second partial leg support frame; and an additional drive module (comprising an actuator and/or circuitry) configured to control a movement of the second partial leg support frame with respect to the first partial leg support frame.

According to an example embodiment, a method of controlling a wearable device performed by the wearable device may include: determining whether a walking state of a user of the wearable device is a normal state based on test movement information of the user obtained through test walking; determining first correction torque information based on the test movement information, when the walking state is not the normal state, wherein the first correction torque information includes a control signal for at least one of a drive module or an additional drive module of the wearable device; and outputting a first correction torque corresponding to the first correction torque information through at least one of the drive module or the additional drive module.

Other features and aspects will be apparent from the following detailed description, the drawings, and the claims.

### BRIEF DESCRIPTION OF THE DRAWINGS

The above and other aspects, features, and advantages of certain example embodiments will be more apparent from the following detailed description, taken in conjunction with the accompanying drawings, in which:

FIG. 1 is a diagram illustrating an overview of a wearable device worn on a body of a user according to an example embodiment;

FIG. 2 is a diagram illustrating an exercise management system including a wearable device and an electronic device according to an example embodiment;

FIG. 3 is a rear view of a wearable device according to an example embodiment;

FIG. 4 is a left side view of a wearable device according to an example embodiment;

FIGS. 5A and 5B are diagrams illustrating example configurations of a control system of a wearable device according to an example embodiment(s);

FIG. 6 is a diagram illustrating an interaction between a wearable device and an electronic device according to an example embodiment;

FIG. 7 is a diagram illustrating a configuration of an electronic device according to an example embodiment;

FIG. 8A is a diagram illustrating a leg support frame including a first partial leg support frame and a second partial leg support frame according to an example embodiment;

FIG. 8B is a diagram illustrating an additional drive module configured to control a movement of a second partial leg support frame with respect to a first partial leg support frame according to an example embodiment;

FIG. 9 is a flowchart illustrating a method of outputting a first correction torque for correcting a walking posture of a user according to an example embodiment;

FIG. 10 is a diagram illustrating a method of obtaining straight leg movement information of a user according to an example embodiment;

FIG. 11 is a diagram illustrating a method of obtaining test pelvic movement information of a user according to an example embodiment;

FIG. 12 is a diagram illustrating a method of obtaining lateral leg movement information of a user according to an example embodiment;

FIG. 13 is a flowchart illustrating a method of determining whether a walking state of a user is a normal state based on a deviation between a test movement range and a reference movement range according to an example embodiment;

FIG. 14 is a flowchart illustrating a method of executing a muscular strength-strengthening exercise program based on a first corrected movement range and a test movement range according to an example embodiment;

FIG. 15 is a flowchart illustrating a method of executing a muscular strength-assisting exercise program in a case in which a walking state of a user is a normal state according to an example embodiment;

FIG. 16A is a flowchart illustrating a method of executing a muscular strength-assisting exercise program according to an example embodiment;

FIG. 16B is a diagram illustrating a gain value of an operation protocol of a walking assistance mode that changes over time according to an example embodiment;

FIG. 17 is a flowchart illustrating a method of outputting a second correction torque for correcting a walking posture of a user according to an example embodiment;

FIG. 18A is a flowchart illustrating a method of executing a muscular strength-strengthening exercise program according to an example embodiment;

FIGS. 18B to 18D are diagrams illustrating a gain value of an operation protocol of a cardiopulmonary strengthening mode that changes over time according to an example embodiment(s);

FIGS. 18E to 18G are diagrams illustrating a gain value of an operation protocol of a muscle strengthening mode that changes over time according to an example embodiment(s); and

FIGS. 18H to 18J are diagrams illustrating a gain value of an operation protocol of an interval training mode that changes over time according to an example embodiment(s).

### DETAILED DESCRIPTION

Hereinafter, various example embodiments will be described with reference to the accompanying drawings. However, the example embodiments are not intended to limit the present disclosure, but various changes, modifications, equivalents, and/or alternatives of the embodiments will be apparent after an understanding of the disclosure.

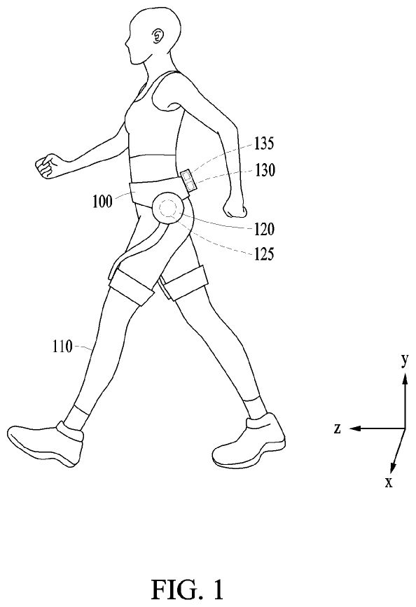

FIG. 1 is a diagram illustrating an overview of a wearable device worn on a body of a user according to an example embodiment.

Referring to FIG. 1, according to an example embodiment, a wearable device 100 may be a device worn on a body of a user 110 to assist the user 110 in walking, exercising, and/or working. In an example embodiment, the wearable device 100 may also be used to measure a physical ability (e.g., a walking ability, an exercise ability, and an exercise posture) of the user 110. In some example embodiments, the term “wearable device” may be replaced with a “wearable robot,” a “walking assistance device,” or an “exercise assistance device.” The user 110 may be a human or an animal but is not limited thereto. The wearable device 100 may be worn on the body of the user 110 (e.g., a lower body (legs, ankles, knees, etc.), an upper body (torso, arms, wrists, etc.), or a waist) to apply an external force such as an assistance force and/or a resistance force to a body movement or motion of the user 110. The assistance force, which is a force applied in the same direction as a body movement of the user 110, may be a force that assists the user 110 with the body movement. The resistance force, which is a force applied in an opposite direction of a body movement of the user 110, may be a force that hinders the body movement of the user 110. The resistance force may also be referred to as an exercise load.

In an example embodiment, the wearable device 100 may operate in a walking assistance mode for assisting the user 110 in walking. In the walking assistance mode, the wearable device 100 may assist the user 110 in walking by applying an assistance force generated from a drive module 120 of the wearable device 100 to the body of the user 110. The wearable device 100 may provide a force required for the user 110 to walk to allow the user 110 to walk independently or for a long time, thereby expanding the walking ability of the user 110. The wearable device 100 may contribute to improving a gait of a pedestrian who has an abnormal walking habit or walking posture.

In an example embodiment, the wearable device 100 may operate in an exercise assistance mode for enhancing an exercise effect for the user 110. In the exercise assistance mode, the wearable device 100 may hinder a body movement of the user 110 or provide resistance to the body movement of the user 110 by applying a resistance force generated from the drive module 120 to the body of the user 110. In a case in which the wearable device 100 is a hip-type wearable device worn on the waist (or pelvis) and legs (e.g., thighs) of the user 110, the wearable device 100 may provide an exercise load to a leg movement of the user 110 while worn the legs of the user 110 and enhance an exercise effect on the legs of the user 110. In an example embodiment, the wearable device 100 may apply the assistance force to the body of the user 110 to assist the user 110 in an exercise performed by the user 110. For example, in a case in which a physically challenged person or elderly attempts to do an exercise with the wearable device 100 on, the wearable device 100 may provide the assistance force to assist the user 110 with a body movement during the exercise. In an example embodiment, the wearable device 100 may combine and provide the assistance force and the resistance force for each exercise period or for each time period, for example, by providing the assistance force in some exercise periods and providing the resistance force in other exercise periods.

In an example embodiment, the wearable device 100 may operate in a physical ability measurement mode for measuring a physical ability of the user 110. In the physical ability measurement mode, the wearable device 100 may measure movement information of the user 110 using sensors (e.g., an angle sensor 125 and an inertial measurement unit (IMU) 135) provided in the wearable device 100 while the user 110 is walking or exercising, and evaluate a physical ability of the user 110 based on the measured movement information. For example, the movement information of the user 110 measured by the wearable device 100 may be used to estimate a walking index or an exercise ability index (e.g., strength, endurance, balance, and exercise posture). The physical ability measurement mode may include an exercise posture measurement mode for measuring an exercise posture of the user 110.

In various example embodiments, for the convenience of description, a hip-type wearable device shown in FIG. 1 is described as an example of the wearable device 100, but examples of which are not limited thereto. As described above, the wearable device 100 may be worn on other body parts (e.g., upper arms, lower arms, hands, calves, and feet) other than the waist and the legs (e.g., the thighs in particular), and may vary in shape and configuration depending on a body part on which the wearable device 100 is worn.

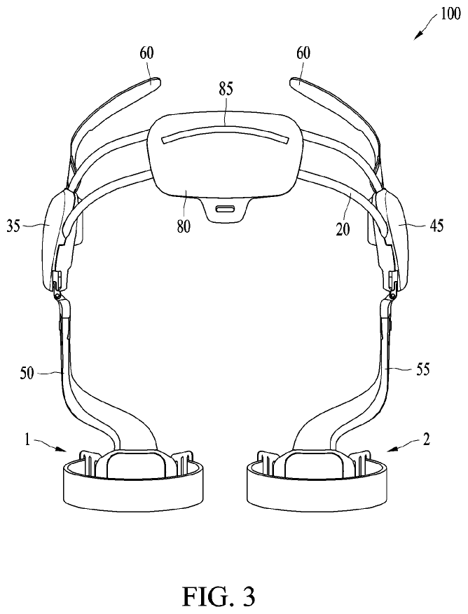

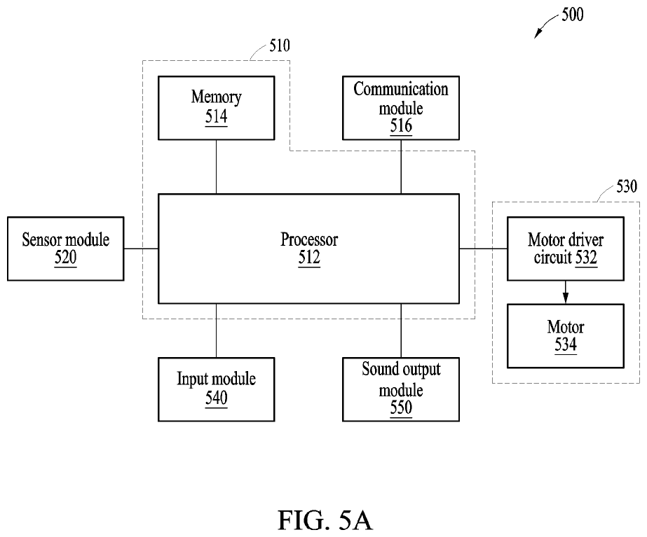

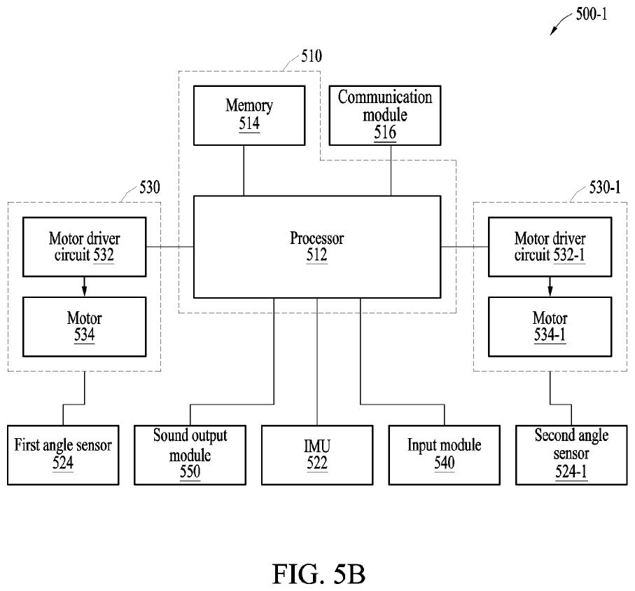

According to an example embodiment, the wearable device 100 may include a support frame (e.g., leg support frames 50 and 55 and a waist support frame 20 of FIG. 3) configured to support the body of the user 110 when the wearable device 100 is worn on the body of the user 110, a sensor module (e.g., a sensor module 520 of FIG. 5A, comprising at least one sensor) configured to obtain sensor data including movement information associated with a body movement (e.g., a leg movement and an upper body movement) of the user 110, the drive module 120 (e.g., drive modules 35 and 45 of FIG. 3, comprising a motor and/or circuitry) configured to generate a torque to be applied to the legs of the user 110, and a control module 130 (e.g., a control module 510 of FIGS. 5A and 5B, comprising processing circuitry) configured to control the wearable device 100.

The sensor module may include the angle sensor 125 and the IMU 135. The angle sensor 125 may measure a rotation angle of a leg support frame (e.g., the leg support frames 50 and 55) of the user 110 corresponding to a hip joint angle value of the user 110. The rotation angle of the leg support frame measured by the angle sensor 125 may be estimated as the hip joint angle value (or a leg angle value) of the user 110. The angle sensor 125 may include, for example, an encoder and/or a Hall sensor. In an example embodiment, the angle sensor 125 may be disposed near a left hip joint and a right hip joint each of the user 110. The IMU 135 may include an acceleration sensor and/or an angular velocity sensor, and may measure a change in acceleration and angular speed by a movement of the user 110. For example, the IMU 135 may measure an upper body movement value of the user 110 corresponding to a movement value of a waist support frame (e.g., the waist support frame 20) (or a base body (e.g., a base body 80 of FIG. 3)). The movement value of the waist support frame measured by the IMU 135 may be estimated as the upper body movement value of the user 110.

In an example embodiment, the control module 130 and the IMU 135 may be disposed in the base body (e.g., the base body 80 of FIG. 3) of the wearable device 100. The base body may be positioned on (or around) a lower back (or a waist) of the user 110 in a state in which the wearable device 100 is worn on the user 110. The base body may be formed on or attached to the outside of the waist support frame of the wearable device 100. The base body, worn on the lower back of the user 110, may provide cushioning to the waist of the user 110 and support the waist of the user 110 together with the waist support frame.

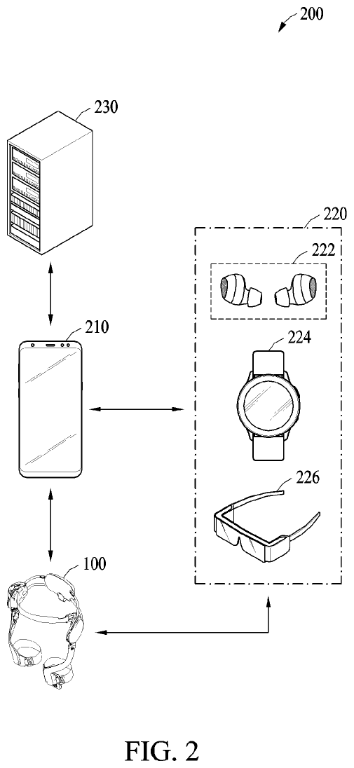

FIG. 2 is a diagram illustrating an exercise management system including a wearable device and an electronic device according to an example embodiment.

Referring to FIG. 2, an exercise management system 200 may include a wearable device 100 to be worn on a body of a user, an electronic device 210, another wearable device 220, and a server 230. In an example embodiment, at least one of these devices (e.g., the other wearable device 220 or the server 230) may be omitted from or at least one other device (e.g., a dedicated controller device for the wearable device 100) may be added to the exercise management system 200.

In an example embodiment, in a walking assistance mode, the wearable device 100 may assist the user with a movement while worn on the body of the user. For example, the wearable device 100 may assist the user in walking by generating an assistance force for assisting the user with a leg movement while worn on the legs of the user.

In an example embodiment, in an exercise assistance mode, the wearable device 100 may generate a resistance force for hindering a body movement of the user or an assistance force for assisting the user with a body movement and apply the resistance force or the assistance force to the body of the user to enhance an exercise effect on the user. For example, in the exercise assistance mode, the user may select, through the electronic device 210, an exercise program (e.g., squat, split lunge, dumbbell squat, knee-up lunge, stretching, etc.) with which the user attempts to do an exercise using the wearable device 100, and/or an exercise intensity to be applied to the wearable device 100. The wearable device 100 may control a drive module of the wearable device 100 according to the exercise program selected by the user and obtain sensor data including movement information of the user through a sensor module. The wearable device 100 may adjust the strength of the resistance force or the assistance force to be applied to the user according to the exercise intensity selected by the user. For example, the wearable device 100 may control the drive module to generate a resistance force corresponding to the exercise intensity selected by the user.

In an example embodiment, the wearable device 100 may be used to measure a physical ability of the user through interworking with the electronic device 210. The wearable device 100 may operate in a physical ability measurement mode which is a mode for measuring a physical ability of the user under the control of the electronic device 210 and may transmit sensor data obtained from a movement of the user in the physical ability measurement mode to electronic device 210. The electronic device 210 may then estimate the physical ability of the user by analyzing the sensor data received from the wearable device 100.

The electronic device 210 may communicate with the wearable device 100, and remotely control the wearable device 100 or provide the user with state information associated with a state (e.g., a booting state, a charging state, a sensing state, and an error state) of the wearable device 100. The electronic device 210 may receive the sensor data obtained by a sensor of the wearable device 100 from the wearable device 100 and estimate a physical ability of the user or a result of an exercise performed by the user based on the received sensor data. In an example embodiment, when the user is doing an exercise with the wearable device 100 worn on the user, the wearable device 100 may obtain sensor data including movement information of the user using sensors and transmit the obtained sensor data to the electronic device 210. The electronic device 210 may extract a movement value of the user from the sensor data and evaluate an exercise posture of the user based on the extracted movement value. The electronic device 210 may provide the user with an exercise posture measurement value and exercise posture evaluation information associated with the exercise posture of the user through a graphical user interface (GUI).

In an example embodiment, the electronic device 210 may execute a program (e.g., an application) for controlling the wearable device 100, and the user may adjust, through the program, operations or setting values (e.g., an intensity of torque output from the drive module (e.g., drive modules 35 and 45 of FIG. 3), a size of audio output from a sound output module (e.g., a sound output module 550 of FIGS. 5A and 5B), and a brightness of a lighting unit (e.g., a lighting unit 85 of FIG. 3)) of the wearable device 100. The program executed on the electronic device 210 may provide a GUI for an interaction with the user. The electronic device 210 may be a device in one of various type. The electronic device 210 may include, as non-limiting examples, a portable communication device (e.g., a smartphone), a computer device, an access point, a portable multimedia device, or a home appliance (e.g., a television (TV), an audio device, and a projector device).

According to an example embodiment, the electronic device 210 may be connected to the server 230 using short-range wireless communication or cellular communication. The server 230 may receive user profile information of the user using the wearable device 100 from the electronic device 210 and store and manage the received user profile information. The user profile information may include, for example, information about at least one of name, age, gender, height, weight, or body mass index (BMI) of the user. The server 230 may receive, from the electronic device 210, exercise history information about an exercise performed by the user, and store and manage the received exercise history information. The server 230 may provide the electronic device 210 with various exercise programs or physical ability measurement programs that may be provided to the user.

According to an example embodiment, the wearable device 100 and/or the electronic device 210 may be connected to the other wearable device 220. The other wearable device 220 may include, as non-limiting examples, wireless earphones 222, a smartwatch 224, or smart glasses 226. In an example embodiment, the smartwatch 224 may measure a biosignal including heart rate information of the user and transmit the measured biosignal to the electronic device 210 and/or the wearable device 100. The electronic device 210 may estimate the heart rate information (e.g., current heart rate, maximum heart rate, and average heart rate) of the user based on the biosignal received from the smartwatch 224 and provide the estimated heart rate information to the user.

In an example embodiment, the exercise result information, physical ability information, and/or exercise posture evaluation information that is evaluated through the electronic device 210 may be transmitted to the other wearable device 220 to be provided to the user through the other wearable device 220. The state information of the wearable device 100 may also be transmitted to the other wearable device 220 to be provided to the user through the other wearable device 220. In an example embodiment, the wearable device 100, the electronic device 210, and the other wearable device 220 may be connected to each other through wireless communication (e.g., Bluetooth communication and wires-fidelity (Wi-Fi) communication).

In an example embodiment, the wearable device 100 may provide (or output) feedback (e.g., visual feedback, auditory feedback, and/or tactile feedback) corresponding to the state of the wearable device 100 according to a control signal received from the electronic device 210. For example, the wearable device 100 may provide visual feedback through the lighting unit (e.g., the lighting unit 85 of FIG. 3) and auditory feedback through the sound output module (e.g., the sound output module 550 of FIGS. 5A and 5B). The wearable device 100 may include a haptic module and provide tactile feedback in the form of vibration to the body of the user through the haptic module. The electronic device 210 may also provide (or output) feedback (e.g., visual feedback, auditory feedback, and/or tactile feedback) corresponding to the state of the wearable device 100.

In an example embodiment, the electronic device 210 may present a personalized exercise goal to the user in the exercise assistance mode. The personalized exercise goal may include a target exercise amount for each exercise type (e.g., a muscle strengthening exercise (or weight exercise), a balance exercise, an aerobic exercise (or cardio exercise)) that the user attempts to do, which may be determined by the electronic device 210 and/or the server 230. When the server 230 determines the target exercise amount, the server 230 may transmit information about the determined target exercise amount to the electronic device 210. The electronic device 210 may then personalize and present a target exercise amount for an exercise type (e.g., the muscle strengthening exercise, the balance exercise, and the aerobic exercise) according to an exercise program (e.g., squat, split lunge, and knee-up lunge) the user attempts to perform and/or physical characteristics (e.g., age, height, weight, and BMI) of the user. The electronic device 210 may display, on a display, a GUI screen that displays the target exercise amount for each exercise type.

In an example embodiment, the electronic device 210 and/or the server 230 may include a database (DB) in which information about a plurality of exercise programs to be provided to the user through the wearable device 100 is stored. To achieve the exercise goal for the user, the electronic device 210 and/or the server 230 may recommend an exercise program that is suitable for the user. The exercise goal may include, for example, at least one of improving muscular strength, improving muscular physical strength, improving cardiovascular endurance, improving core stability, improving flexibility, or improving symmetry. The electronic device 210 and/or the server 230 may store and manage the exercise program performed by the user, a result of performing the exercise program, and the like.

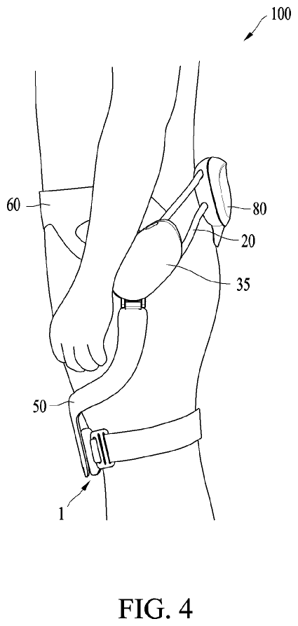

FIG. 3 is a rear view of a wearable device according to an example embodiment, and FIG. 4 is a left side view of a wearable device according to an example embodiment.

According to an example embodiment, referring to FIGS. 3 and 4, the wearable device 100 may include a base body 80, a waist support frame 20, drive modules 35 and 45 each comprising a motor and/or circuitry (e.g., see 530 and 530-1), leg support frames 50 and 55, thigh fasteners 1 and 2, and a waist fastener 60. The base body 80 may include a lighting unit 85. In an example embodiment, at least one of these components (e.g., the lighting unit 85) may be omitted from or at least one other component (e.g., a haptic module) may be added to the wearable device 100.

The base body 80, which may comprise a housing, may be positioned on (or around) a lower back or waist of a user while the wearable device 100 is worn on a body of the user. The base body 80 may be positioned on the lower back of the user to provide a cushioning feeling to the waist of the user and support the waist of the user. The base body 80 may be hung around buttocks of the user such that the wearable device 100 does not escape downward by gravity while the wearable device 100 is worn on the user. The base body 80 may distribute a portion of the weight of the wearable device 100 to the waist of the user while the wearable device 100 is worn on the user. The base body 80 may be connected, directly or indirectly, to the waist support frame 20. At both ends of the base body 80, a waist support frame connection element (not shown) that may be connected, directly or indirectly, to the waist support frame 20 may be provided.

In an example embodiment, the lighting unit 85 may be disposed outside the base body 80. The lighting unit 85 may include a light source (e.g., a light-emitting diode (LED)). The lighting unit 85 may emit light under the control of a control module (not shown) (e.g., a control module 510 of FIGS. 5A and 5B). Depending on an example embodiment, the control module may control the lighting unit 85 to provide (or output) visual feedback corresponding to a state of the wearable device 100 to the user through the lighting unit 85.

The waist support frame 20 may extend from both ends of the base body 80. Inside the waist support frame 20, the waist of the user may be accommodated. The waist support frame 20 may include at least one rigid body beam. Each beam may be provided in a curved shape having a preset curvature to surround the waist of the user. The waist fastener 60 may be connected, directly or indirectly, to an end of the waist support frame 20. The drive modules 35 and 45 may be connected, directly or indirectly, to the waist support frame 20.

In an example embodiment, the control module, an IMU (not shown) (e.g., the IMU 135 of FIG. 1 and an IMU 522 of FIG. 5B), a communication module (not shown) (e.g., a communication module 516 of FIGS. 5A and 5B, comprising communication circuitry), and a battery (not shown) may be disposed inside the base body 80. The base body 80 may protect the control module, the IMU, the communication module, and the battery. The control module may generate a control signal for controlling an operation of the wearable device 100. The control module may include a control circuit including at least one processor and a memory to control actuators of the drive modules 35 and 45. The control module may further include a power supply module (not shown) to supply power of the battery to each of the components of the wearable device 100.

In an example embodiment, the wearable device 100 may include a sensor module (not shown) (e.g., a sensor module 520 of FIG. 5A) configured to obtain sensor data from at least one sensor. The sensor module may obtain the sensor data that changes according to a movement of the user. In an example embodiment, the sensor module may obtain the sensor data including movement information of the user and/or movement information of a component of the wearable device 100. The sensor module may include, as non-limiting examples, an IMU (e.g., the IMU 135 of FIG. 1 or the IMU 522 of FIG. 5B) for measuring an upper body movement value of the user or a movement value of the waist support frame 20, and an angle sensor (e.g., the angle sensor 125 of FIG. 1, and a first angle sensor 524 and a second angle sensor 524-1 of FIG. 5B) for measuring a hip joint angle value of the user or a movement value of the leg support frames 50 and 55. The sensor module may further include, for example, at least one of a position sensor, a temperature sensor, a biosignal sensor, or a proximity sensor.

The waist fastener 60 may be connected, directly or indirectly, to the waist support frame 20 to fasten the waist support frame 20 to the waist of the user. The waist fastener 60 may include, for example, a pair of belts.

The drive modules 35 and 45 may generate an external force (or torque) to be applied to the body of the user based on the control signal generated by the control module. For example, the drive modules 35 and 45 may generate an assistance force or a resistance force to be applied to the legs of the user. In an example embodiment, the drive modules 35 and 45 may include a first drive module 45 disposed at a position corresponding to a position of a right hip joint of the user and a second drive module 35 disposed at a position corresponding to a position of a left hip joint of the user. The first drive module 45 may include a first actuator and a first joint member, and the second drive module 35 may include a second actuator and a second joint member. The first actuator may provide power to be transmitted to the first joint member, and the second actuator may provide power to be transmitted to the second joint member. The first actuator and the second actuator may each include a motor configured to generate power (or torque) by receiving power from the battery. When powered and driven, the motor may generate a force (e.g., the assistance force) for assisting a physical movement of the user or a force (e.g., the resistance force) for hindering a physical movement of the user. In an example embodiment, the control module may adjust a voltage and/or current to be supplied to the motor to adjust the intensity and direction of the force to be generated by the motor.

In an example embodiment, the first joint member and the second joint member may receive power from the first actuator and the second actuator, respectively, and may apply an external force to the body of the user based on the received power. The first joint member and the second joint member may be disposed at corresponding positions of joint portions of the user, respectively. One side of the first joint member may be connected, directly or indirectly, to the first actuator, and the other side thereof may be connected, directly or indirectly, to a first leg support frame 55. The first joint member may be rotated by the power received from the first actuator. An encoder or a Hall sensor that may operate as the angle sensor for measuring a rotation angle (corresponding to a joint angle of the user) of the first joint member may be disposed on one side of the first joint member. One side of the second joint member may be connected, directly or indirectly, to the second actuator, and the other side thereof may be connected, directly or indirectly, to a second leg support frame 50. The second joint member may be rotated by the power received from the second actuator. An encoder or a Hall sensor that may operate as the angle sensor for measuring a rotation angle (corresponding to a joint angle of the user) of the second joint member may be disposed on one side of the second joint member.

In an example embodiment, the first actuator may be disposed in a lateral direction of the first joint member, and the second actuator may be disposed in a lateral direction of the second joint member. A rotation axis of the first actuator and a rotation axis of the first joint member may be disposed to be separate from each other, and a rotation axis of the second actuator and a rotation axis of the second joint member may also be disposed to be separate from each other. However, examples are not limited thereto, and each actuator and each joint member may share a rotation axis. In an example embodiment, each actuator may be disposed to be separate from each joint member. In this case, the drive modules 35 and 45 may further include a power transmission module (not shown) configured to transmit power from the respective actuators to the respective joint members. The power transmission module may be a rotary body (e.g., a gear), or a longitudinal member (e.g., a wire, a cable, a string, a spring, a belt, or a chain). However, the scope of examples is not limited to the foregoing positional relationship between the actuators and the joint members, and the foregoing power transmission structure.

In an example embodiment, the leg support frames 50 and 55 may support the legs (e.g., thighs) of the user when the wearable device 100 is worn on the legs of the user. For example, the leg support frames 50 and 55 may transmit power (e.g., torque) generated by the drive modules 35 and 45 to the thighs of the user, and the power may act as an external force to be applied to a movement of the legs of the user. As one end of the leg support frames 50 and 55 is connected, directly or indirectly, to a joint member to be rotated and the other end of the leg support frames 50 and 55 is connected, directly or indirectly, to the thigh fasteners 1 and 2, the leg support frames 50 and 55 may transmit the power generated by the drive modules 35 and 45 to the thighs of the user while supporting the thighs of the user. For example, the leg support frames 50 and 55 may push or pull the thighs of the user. The leg support frames 50 and 55 may extend in a longitudinal direction of the thighs of the user. The leg support frames 50 and 55 may be bent to wrap at least a portion of the circumference of the thighs of the user. The leg support frames 50 and 55 may include the first leg support frame 55 for supporting the right leg of the user and the second leg support frame 50 for supporting the left leg of the user.

The thigh fasteners 1 and 2 may be connected, directly or indirectly, to the leg support frames 50 and 55 and may attach (or, fix) the leg support frames 50 and 55 to the thighs of the user. The thigh fasteners 1 and 2 may include a first thigh fastener 2 for fixing the first leg support frame 55 to the right thigh of the user, and a second thigh fastener 1 for fixing the second leg support frame 50 to the left thigh of the user.

In an example embodiment, the first thigh fastener 2 may include a first cover, a first fastening frame, and a first strap. The second thigh fastener 1 may include a second cover, a second fastening frame, and a second strap. The first cover and the second cover may apply torque generated by the drive modules 35 and 45 to the thighs of the user. The first cover and the second cover may be disposed on one side of the thighs of the user to push or pull the thighs of the user. The first cover and the second cover may be disposed on a front surface of the thighs of the user. The first cover and the second cover may be disposed along a circumferential direction of the thighs of the user. The first cover and the second cover may extend to both sides around the other ends of the leg support frames 50 and 55 and may include curved surfaces corresponding to the thighs of the user. One ends of the first cover and the second cover may be connected, directly or indirectly, to corresponding fastening frames, and the other ends thereof may be connected, directly or indirectly, to corresponding straps.

For example, the first fastening frame and the second fastening frame may be disposed to surround at least a portion of the circumference of the thighs of the user to prevent or reduce a chance of the thighs of the user from escaping from the leg support frames 50 and 55. The first fastening frame may have a fastening structure that connects the first cover and the first strap, and the second fastening frame may have a fastening structure that connects the second cover and the second strap.

The first strap may surround a remaining portion of the circumference of the right thigh of the user that is not covered by the first cover and the first fastening frame, and the second strap may surround a remaining portion of the circumference of the left thigh of the user that is not covered by the second cover and the second fastening frame. The first strap and the second strap may each include, for example, an elastic material (e.g., a band).

FIGS. 5A and 5B are diagrams illustrating example configurations of a control system of a wearable device according to an example embodiment.

Referring to FIG. 5A, the wearable device 100 may be controlled by a control system 500. The control system 500 may include a control module 510, a communication module 516, a sensor module 520, a drive module 530, an input module 540, and a sound output module 550. In an example embodiment, at least one of these components (e.g., the sound output module 550) may be omitted from or at least one other component (e.g., a haptic module) may be added to the control system 500.

The drive module 530 may include a motor 534 configured to generate power (e.g., torque) and a motor driver circuit 532 configured to drive the motor 534. Although a drive module (e.g., the drive module 530) is illustrated as including a single motor driver circuit (e.g., the motor driver circuit 532) and a single motor (e.g., the motor 534) in FIG. 5A, examples of which are not limited thereto. For example, as shown in FIG. 5B, a drive module (e.g., a drive module 530-1) of a control system 500-1 may include a plurality of (e.g., two or more) motor driver circuits (e.g., motor driver circuits 532 and 532-1) and motors (e.g., motors 534 and 534-1). The drive module 530 including the motor driver circuit 532 and the motor 534 may correspond to the first drive module 45 of FIG. 3, and the drive module 530-1 including the motor driver circuit 532-1 and the motor 534-1 may correspond to the second drive module 35 of FIG. 3. The following description of each of the motor driver circuit 532 and the motor 534 may also be applied to the motor driver circuit 532-1 and the motor 534-1 shown in FIG. 5B.

Referring back to FIG. 5A, the sensor module 520 may include a sensor circuit including at least one sensor. The sensor module 520 may include sensor data including movement information of a user or movement information of the wearable device 100. The sensor module 520 may transmit the obtained sensor data to the control module 510. The sensor module 520 may include an IMU 522 and an angle sensor (e.g., a first angle sensor 524 and a second angle sensor 524-1), as shown in FIG. 5B. The IMU 522 may measure an upper body movement value of the user. For example, the IMU 522 may sense X-axis, Y-axis, and Z-axis acceleration, and sense X-axis, Y-axis, and Z-axis angular velocity according to a movement of the user. The IMU 522 may be used to measure at least one of, for example, a forward and backward tilt, a leftward and rightward tilt, or a rotation of the body of the user. In addition, the IMU 522 may obtain a movement value (e.g., an acceleration value and an angular velocity value) of a waist support frame (e.g., the waist support frame 20 of FIG. 3) of the wearable device 100. The movement value of the waist support frame may correspond to the upper body movement value of the user.

The angle sensor may measure a hip joint angle value of the user according to a movement of the legs of the user. The sensor data that may be measured by the angle sensor may include, for example, a hip joint angle value of a right leg, a hip joint angle value of a left leg, and information about a direction of a movement of the legs. For example, the first angle sensor 524 of FIG. 5B may obtain the hip joint angle value of the right leg of the user, and the second angle sensor 524-1 may obtain the hip joint angle value of the left leg of the user. The first angle sensor 524 and the second angle sensor 524-1 may each include an encoder and/or a Hall sensor, for example. The angle sensor may also obtain a movement value of a leg support frame of the wearable device 100. For example, the first angle sensor 524 may obtain a movement value of the first leg support frame 55, and the second angle sensor 524-1 may obtain a movement value of the second leg support frame 50. The movement value of the leg support frame may correspond to the hip joint angle value.

In an example embodiment, the sensor module 520 may further include at least one of a position sensor for obtaining a position value of the wearable device 100, a proximity sensor for detecting proximity of an object, a biosignal sensor for detecting a biosignal of the user, or a temperature sensor for measuring an ambient temperature.

The input module 540 may receive a command or data to be used by a component (e.g., a processor(s) 512) of the wearable device 100 from the outside (e.g., the user) of the wearable device 100. The input module 540 may include an input component circuit. The input module 540 may include, for example, a key (e.g., a button) or a touchscreen.

The sound output module 550 may output a sound signal to the outside of the wearable device 100. The sound output module 550 may provide auditory feedback to the user. For example, the sound output module 550 may include a speaker that reproduces a guide voice for an audible notification of a guide sound signal (e.g., a driving start sound, a posture error notification sound, or an exercise start notification sound), music content, or specific information (e.g., exercise result information and exercise posture evaluation information).

In an example embodiment, the control system 500 may further include a battery (not shown) for supplying power to each component of the wearable device 100. The wearable device 100 may convert power of the battery according to an operating voltage of each component of the wearable device 100 and supply the converted power to each component.

The drive module 530 may generate an external force to be applied to the legs of the user under the control of the control module 510. The drive module 530 may generate a torque to be applied to the legs of the user based on a control signal generated by the control module 510. The control module 510 may transmit the control signal to the motor driver circuit 532. The motor driver circuit 532 may control an operation of the motor 534 by generating a current signal (or a voltage signal) corresponding to the control signal and supplying the generated current signal to the motor 534. The current signal may not be supplied to the motor 534, as needed. When the motor 534 is driven as the current signal is supplied to the motor 534, the motor 534 may generate a torque for an assistance force for assisting a movement of the legs of the user or a resistance force for hindering a movement of the legs of the user.

The control module 510 may control an overall operation of the wearable device 100 and may generate a control signal for controlling each component (e.g., the communication module 516 and the drive module 530). The control module 510 may include at least one processor 512 and a memory 514.

For example, the at least one processor 512 may execute software to control at least one other component (e.g., a hardware or software component) of the wearable device 100 connected, directly or indirectly, to the processor 512 and may perform various types of data processing or computation. The software may include an application for providing a GUI. According to an example embodiment, as at least a part of data processing or computation, the processor 512 may store instructions or data received from another component (e.g., the communication module 516) in the memory 514, process the instructions or data stored in the memory 514, and store resulting data obtained by the processing in the memory 514. According to an example embodiment, the processor 512 may include, for example, one or more of a main processor (e.g., a central processing unit (CPU) or an application processor (AP)) or an auxiliary processor (e.g., a graphics processing unit (GPU), a neural processing unit (NPU), an image signal processor (ISP), a sensor hub processor, or a communication processor (CP)) that is operable independently of, or in conjunction with, the main processor. The auxiliary processor may be implemented separately from the main processor or as a part of the main processor.

The memory 514 may store various pieces of data used by at least one component (e.g., the processor 512) of the control module 510. The data may include, for example, input data or output data for software, sensor data, and instructions related thereto. The memory 514 may include a volatile memory or a non-volatile memory (e.g., a random-access memory (RAM), a dynamic RAM (DRAM), or a static RAM (SRAM)).

The communication module 516 may support establishment of a direct (e.g., wired) communication channel or a wireless communication channel between the control module 510 and another component of the wearable device 100 or an external electronic device (e.g., the electronic device 210 or the other wearable device 220 of FIG. 2), and support communication through the established communication channel. The communication module 516 may include a communication circuit for performing a communication function. For example, the communication module 516 may receive a control signal from an electronic device (e.g., the electronic device 210) and transmit the sensor data obtained by the sensor module 520 to the electronic device. The communication module 516 may include at least one CP (not shown) that is operable independently of the processor 512 and that supports the direct (e.g., wired) communication or the wireless communication. According to an example embodiment, the communication module 516 may include a wireless communication module (e.g., a cellular communication module, a short-range wireless communication module, or a global navigation satellite system (GNSS) communication module), and/or a wired communication module. A corresponding one of these communication modules may communicate with another component of the wearable device 100 and/or an external electronic device via a short-range communication network (e.g., Bluetooth™, Wi-Fi, or infrared data association (IrDA)), or a long-range communication network (e.g., a legacy cellular network, a 5G network, a next-generation communication network, the Internet, or a computer network (e.g., a local area network (LAN) or a wide area network (WAN)).

In an example embodiment, the control system (e.g., the control systems 500 and 500-1) may further include a haptic module (not shown). The haptic module may provide tactile feedback to the user under the control of the processor 512. The haptic module may convert an electrical signal into a mechanical stimulus (e.g., a vibration or a movement) or an electrical stimulus that may be recognized by the user via their tactile sensation or kinesthetic sensation. The haptic module may include, for example, a motor, a piezoelectric element, or an electrical stimulation device. In an example embodiment, the haptic module may be positioned on at least one of a base body (e.g., the base body 80), a first thigh fastener (e.g., the first thigh fastener 2), or a second thigh fastener (e.g., the second thigh fastener 1).

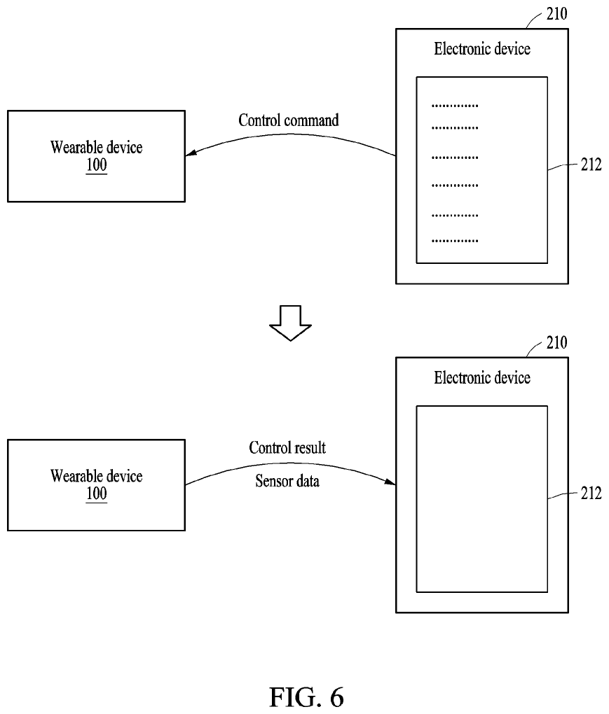

FIG. 6 is a diagram illustrating an interaction between a wearable device and an electronic device according to an example embodiment.

Referring to FIG. 6, the wearable device 100 may communicate with the electronic device 210. For example, the electronic device 210 may be a user terminal of a user who uses the wearable device 100, or a dedicated controller for the wearable device 100. According to an example embodiment, the wearable device 100 and the electronic device 210 may be connected to each other through short-range wireless communication (e.g., Bluetooth communication or Wi-Fi communication).

In an example embodiment, the electronic device 210 may execute an application for checking a state of the wearable device 100 or controlling or operating the wearable device 100. When the application is executed, a screen of a user interface (UI) for controlling an operation of the wearable device 100 or determining an operation mode of the wearable device 100 may be displayed on a display 212 of the electronic device 210. The UI may be a graphical user interface (GUI), for example.

In an example embodiment, the user may input a command (e.g., a command for executing a walking assistance mode, an exercise assistance mode, or a physical ability measurement mode) for controlling the operation of the wearable device 100 or change settings of the wearable device 100, through the screen of the GUI on the display 212 of the electronic device 210. The electronic device 210 may generate a control command (or a control signal) corresponding to an operation control command or a settings change command that is input by the user and transmit the generated control command to the wearable device 100. The wearable device 100 may operate according to the received control command and may transmit, to the electronic device 210, a control result obtained in response to the control command and/or sensor data measured by the sensor module of the wearable device 100. The electronic device 210 may provide, to the user through the screen of the GUI, resulting information (e.g., walking ability information, exercise ability information, and exercise posture evaluation information) derived by analyzing the control result and/or the sensor data.

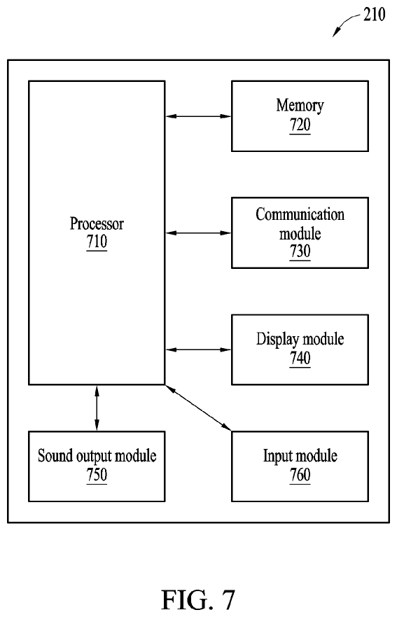

FIG. 7 is a diagram illustrating a configuration of an electronic device according to an example embodiment.

Referring to FIG. 7, the electronic device 210 may include at least one processor 710, a memory 720, a communication module 730 comprising communication circuitry, a display module 740, a sound output module 750 comprising circuitry and/or a speaker, and an input module 760 comprising input circuitry. In an example embodiment, at least one of these components (e.g., the sound output module 750) may be omitted from or at least other component (e.g., a sensor module and a battery) may be added to the electronic device 210.

The at least one processor 710 may control at least one other component (e.g., a hardware or software component) of the electronic device 210 and may perform various types of data processing or computation. According to an example embodiment, as at least a part of data processing or computation, the processor 710 may store instructions or data received from another component (e.g., the communication module 730) in the memory 720, process the instructions or data stored in the memory 720, and store result data in the memory 720.

According to an example embodiment, the processor 710 may include one or more of a main processor (e.g., a CPU or an AP) or an auxiliary processor (e.g., a GPU, an NPU, an ISP, a sensor hub processor, or a CP) that is operable independently of or in conjunction with the main processor.

The memory 720 may store various pieces of data to be used by at least one component (e.g., the processor 710 or the communication module 730) of the electronic device 210. The data may include, for example, a program (e.g., an application), and input data or output data for a command related thereto. The memory 720 may include at least one instruction executable by the processor 710. The memory 720 may include, for example, a volatile memory or a non-volatile memory.

The communication module 730, comprising communication circuitry, may support establishing a direct (e.g., wired) communication channel or a wireless communication channel between the electronic device 210 and another electronic device (e.g., the wearable device 100, the other wearable device 220, or the server 230, such as in FIG. 2) and performing communication via the established communication channel. The communication module 730 may include a communication circuit for performing a communication function. The communication module 730 may include at least one CP that is operable independently of the processor 710 (e.g., an AP) and that supports direct (e.g., wired) communication or wireless communication. According to an example embodiment, the communication module 730 may include a wireless communication module (e.g., a Bluetooth communication module, a cellular communication module, a Wi-Fi communication module, or a GNSS communication module) that performs wireless communication or a wired communication module (e.g., a local area network (LAN) communication module or a power line communication (PLC) module). For example, the communication module 730 may transmit a control command to the wearable device 100 and receive, from the wearable device 100, at least one of sensor data including body movement information of the user wearing the wearable device 100, state data of the wearable device 100, or control result data corresponding to the control command.

The display module 740 may visually provide information to the outside (e.g., the user) of the electronic device 210. The display module 740 may include, for example, a liquid-crystal display (LCD) or an organic light-emitting diode (OLED) display, a hologram device, or a projector. The display module 740 may further include a control circuit for controlling driving of a corresponding display. In an example embodiment, the display module 740 may further include a touch sensor configured to sense a touch or a pressure sensor configured to measure an intensity of a force of the touch.

The sound output module 750 may output a sound signal to the outside of the electronic device 210. The sound output module 750 may include a speaker configured to reproduce a guide sound signal (e.g., a driving start sound an operation error notification sound) based on a state of the wearable device 100, music contents, or a guide voice. For example, when it is determined that the wearable device 100 is not correctly worn on the body of the user, the sound output module 750 may output a guide voice to notify the user of such abnormal wearing or allow the user to wear it normally. For example, the sound output module 750 may output a guide voice corresponding to exercise evaluation information obtained by an evaluation of an exercise performed by the user or exercise result information.

The input module 760 may receive, from the outside (e.g., the user) of the electronic device 210, a command or data to be used by another component (e.g., the processor 710) of the electronic device 210. The input module 760 may include an input component circuit and receive a user input from the user. The input module 760 may include, for example, a key (e.g., a button) or a touchscreen.

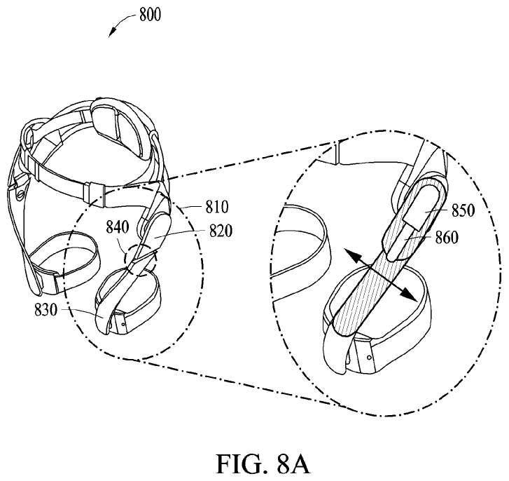

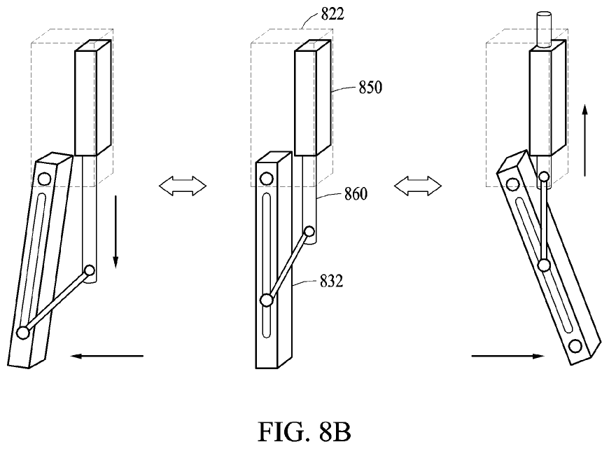

FIG. 8A is a diagram illustrating a leg support frame including a first partial leg support frame and a second partial leg support frame according to an example embodiment.

According to an example embodiment, a wearable device 800 (e.g., the wearable device 100 of FIG. 1) may include a leg support frame 810. The leg support frame 810 of the wearable device 800 may include a first partial leg support frame 820 connected, directly or indirectly, to a drive module (e.g., the drive modules 35 and 45 of FIG. 3), a second partial leg support frame 830 connected, directly or indirectly, to a thigh fastener (e.g., the thigh fasteners 1 and 2 of FIG. 3), a hinge 840 connecting the first partial leg support frame 820 and the second partial leg support frame 830, and an additional drive module 850 configured to control a movement of the second partial leg support frame 830 with respect to the first partial leg support frame 820. The additional drive module 850 may include a rod 860 for controlling the movement of the second partial leg support frame 830. The additional drive module 850 may include a linear actuator. The linear actuator may include a motor and the rod 860. The additional drive module 850 may control the movement of the second partial leg support frame 830 connected, directly or indirectly, to the rod 860 by linearly moving the rod 860 using the motor. A method of controlling the movement of the second partial leg support frame 830 using the additional drive module 850 will be described in detail below with reference to FIG. 8B.

According to an example embodiment, the leg support frame 810 may further include an additional angle sensor configured to measure an angle between the first partial leg support frame 820 and the second partial leg support frame 830. For example, the additional angle sensor may be disposed around the hinge 840 to directly measure an angle of the hinge 840. For example, the additional angle sensor may be a sensor that measures a rotation angle of the motor of the additional drive module 850, and the angle between the first partial leg support frame 820 and the second partial leg support frame 830 may be indirectly determined based on the rotation angle of the motor of the additional drive module 850. For example, the additional angle sensor may be a sensor that measures a position of the rod 860 of the additional drive module 850, and the angle between the first partial leg support frame 820 and the second partial leg support frame 830 may be indirectly determined based on the position of the rod 860.

FIG. 8B is a diagram illustrating an additional drive module configured to control a movement of a second partial leg support frame with respect to a first partial leg support frame according to an example embodiment.

According to an example embodiment, the first partial leg support frame 820 may further include a housing 822 including the additional drive module 850. The additional drive module 850 may control the position of the rod 860 using the motor. The rod 860 may be connected, directly or indirectly, to a connection portion 832 connected, directly or indirectly, to the second partial leg support frame 830. A first end of the connection portion 832 may be connected, directly or indirectly, to the housing 822, and a second end of the connection portion 832 may be connected, directly or indirectly, to the second partial leg support frame 830.

When the position of the rod 860 is changed by the motor, a position of the connection portion 832 may also be changed. For example, a center portion of FIG. 8B shows a state in which the rod 860 and the connection portion 832 are arranged side by side, a left portion shows a state in which the second end of the connection portion 832 moves to the left, and a right portion shows a state in which the second end of the connection portion 832 moves to the right. Based on the direction and distance in and by which the second end of the connection portion 832 moves, the angle of the second partial leg support frame 830 with respect to the first partial leg support frame 820 may be changed.

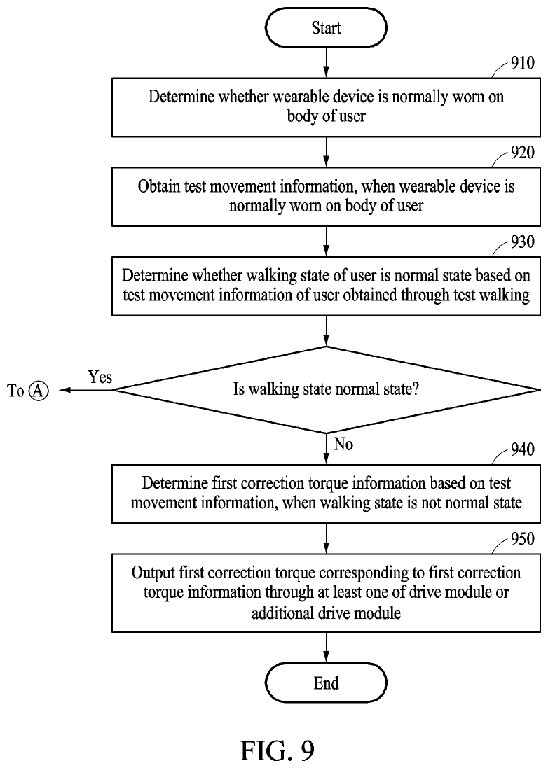

FIG. 9 is a flowchart illustrating a method of outputting a first correction torque for correcting a walking posture of a user according to an example embodiment.

Operations 910 to 950 described below may be performed by a wearable device (e.g., the wearable device 100 of FIG. 1 or the wearable device 800 of FIG. 8).

In operation 910, the wearable device may determine whether the wearable device is normally worn on a body of a user. For example, the wearable device may determine whether a thigh fastener (e.g., the thigh fasteners 1 and 2 such as in FIG. 3) and a waist fastener (e.g., the waist fastener 60 such as in FIG. 3) are normally worn on the body of the user through a wearing detection sensor disposed in each of the thigh fasteners 1 and 2 and the waist fastener 60. For example, the wearing detection sensor may determine whether the thigh fasteners 1 and 2 and the waist fastener 60 are normally worn on the body of the user through a mechanical or electromagnetic method, but an operating method of the wearing detection sensor is not limited to the foregoing example.

According to an example embodiment, when it is determined that any one of the thigh fasteners 1 and 2 and the waist fastener 60 is not normally worn on the body of the user, the wearable device may be determined not to be normally worn on the body of the user.

In operation 920, when the wearable device is normally worn on the body of the user, the wearable device may obtain test movement information.

According to an example embodiment, the user may perform test walking with the wearable device on, and the wearable device may obtain the test movement information associated with walks of the user through the test walking. The test walking may be performed to check what form the user walks, and information associated with the walks of the user may be obtained as the test movement information. During the test walking, the wearable device may not output a torque to the user.

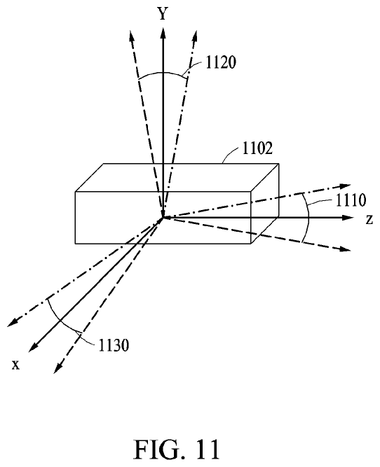

The test movement information may include test pelvic movement information of the user obtained through an IMU (e.g., the IMU 135 of FIG. 1). In this case, pelvic movement information may include angle information and angular velocity information associated with a pelvic movement on an X-axis, a Y-axis, and a Z-axis. The pelvic movement information of the user will be described in detail below with reference to FIG. 11.

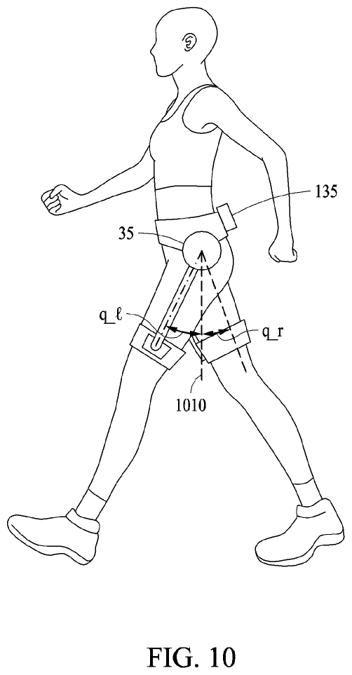

The test movement information may include straight leg movement information of the user obtained through an angle sensor. The straight leg movement information may include maximum/high front angle information and maximum/high rear angle information of a left/right leg of the user. The straight leg movement information will be described in detail below with reference to FIG. 10.

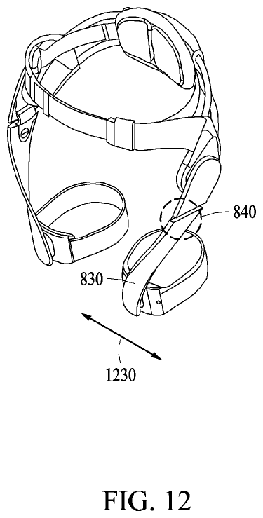

The test movement information may include lateral leg movement information of the user obtained through an additional angle sensor that measures an angle between a first partial leg support frame and a second partial leg support frame of a leg support frame. For example, the lateral leg movement information may include angle information between the first partial leg support frame 820 and the second partial leg support frame 830 described above with reference to FIG. 8. The lateral leg movement information will be described in detail below with reference to FIG. 12.

In operation 930, the wearable device may determine whether a walking state of the user is a normal state based on the test movement information of the user obtained through the test walking.

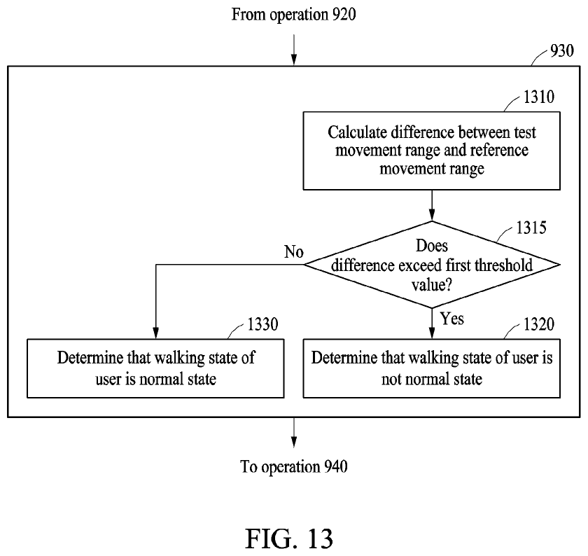

According to an example embodiment, the wearable device may determine whether the walking state of the user is the normal state based on the test movement information and reference movement information. For example, the reference movement information may be information associated with a movement that is shown on average when a person walks normally. A method of determining whether the walking state of the user is the normal state based on the test movement information will be described in detail below with reference to FIG. 13.

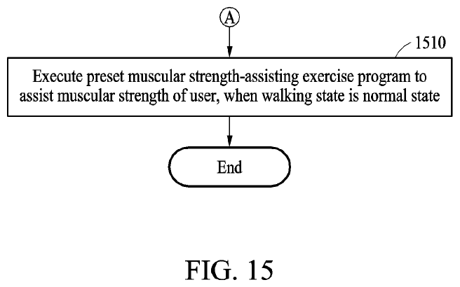

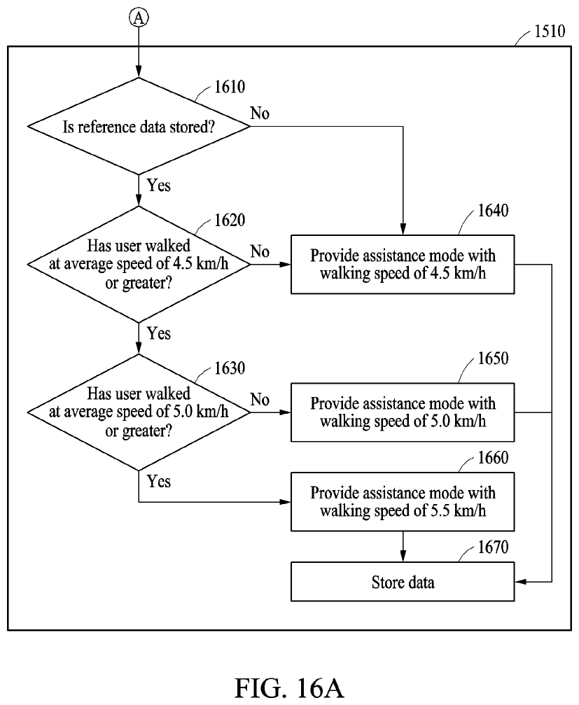

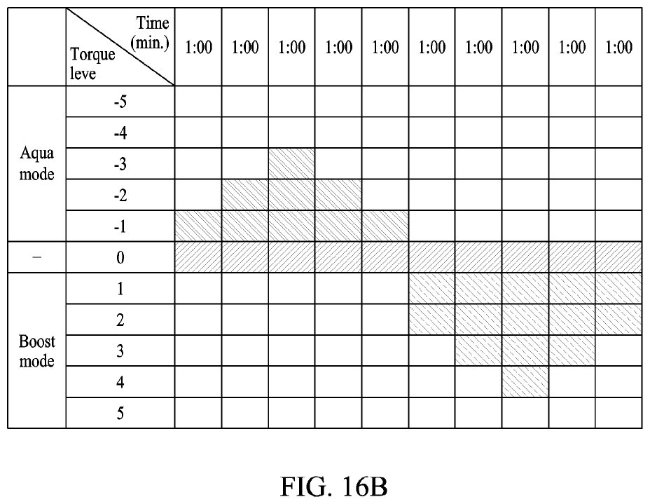

When it is determined that the walking state of the user is the normal state, the following operation A may be performed to provide the user with a muscular strength-assisting exercise program. The operation A will be described in detail below with reference to FIGS. 15 and 16A.

When it is determined that the walking state of the user is not the normal state, operation 940 may be performed.

In operation 940, when the walking state of the user is not the normal state, the wearable device may determine first correction torque information based on the test movement information. For example, the first correction torque information may include a control signal for at least one of a drive module (e.g., the drive module 120 of FIG. 1) or an additional drive module (e.g., the additional drive module 850 of FIG. 8).

According to an example embodiment, the wearable device may determine the first correction torque information based on a difference (or deviation) between a test movement range and a preset reference movement range.

For example, when a range between a maximum front angle and a maximum rear angle of the left/right leg of the user is less than the reference movement range, the first correction torque information may be determined to increase the range between the maximum front angle and the maximum rear angle. For example, when a reference maximum front angle is +30 degrees (°) and a reference maximum rear angle is −15°, the reference movement range may be 45°. In this example, when the test movement range of the user is 30°, the difference between the test movement range and the preset reference movement range may be calculated as 15°. The wearable device may determine the first correction torque information for providing an assistance torque to the user such that the range between the maximum front angle and the maximum rear angle of the leg of the user increases. The wearable device may determine the first correction torque information such that an angle of a preset ratio to the difference between the test movement range and the preset reference movement range is to additionally occur when the user walks. For example, when the difference is 15°, the first correction torque information may be determined such that an angle of 5% of 15° is to additionally occur when the user walks. In this example, 5% described above as the preset ratio is provided only as an example, and the preset ratio is not limited thereto. The first correction torque information for increasing the range between the maximum front angle and the maximum rear angle of the user may be information for controlling the drive module (e.g., the drive module 120 of FIG. 1 or the drive modules 530 and 530-1 of FIGS. 5A and 5B).

For example, when a test pelvic movement range of the user is greater than the reference movement range, the first correction torque information may be determined to reduce the pelvic movement range. For example, when an X-axis angle range of a reference pelvic movement is 10° and an X-axis angle range of a test pelvic movement is 20°, the difference between the test movement range and the preset reference movement range may be calculated as 10°. In this example, the wearable device may determine the first correction torque information for providing an assistance torque to the user such that the pelvic movement range of the user is reduced. For example, when the difference is 10°, the first correction torque information may be determined such that an angle of 5% of 10° is to be reduced when the user walks. In this example, 5% described above as the preset ratio is provided only as an example, and the preset ratio is not limited thereto. The reference movement range of a pelvic movement may be set differently for each of the X-axis, Y-axis, and Z-axis. The first correction torque information for reducing the pelvic movement range of the user may be information for controlling the drive module and the additional drive module (e.g., the additional drive module 850 of FIG. 8).

For example, when an angle range between the first partial leg support frame 820 and the second partial leg support frame 830 is greater than the reference movement range, the first correction torque information may be determined to reduce the angle range between the first partial leg support frame 820 and the second partial leg support frame 830. The first correction torque information for reducing the angle range between the first partial leg support frame 820 and the second partial leg support frame 830 may be information for controlling the additional drive module.

In operation 950, the wearable device may output a first correction torque corresponding to the first correction torque information through at least one of the drive module or the additional drive module. For example, the output first correction torque may be in the form of a torque trajectory corresponding to an entire walking cycle of the user. For example, when the left leg of the user steps forward, a first torque and a first additional torque may be output to assist the left leg in swinging, and when the left leg of the user steps backward, the first torque and the first additional torque may be output to assist the left leg in supporting.

According to an example embodiment, a first torque value of the first correction torque may be calculated using Equations 1 and 2 described later with reference to FIG. 10. For example, in a process of calculating the first torque value, a gain k and a delay Δt for increasing the range between the maximum front angle and the maximum rear angle of the leg of the user may be determined.

According to an example embodiment, the first correction torque may include a first additional torque for controlling the additional drive module to reduce a lateral movement of the leg of the user. For example, to reduce a movement of a leg of the user inward to the torso when the user walks, the first additional torque may be a control signal of the additional drive module to move the second partial leg support frame 830 outward to the torso. The term “first additional torque value” used herein may refer to the magnitude of the first additional torque output at a specific point in time.

According to an example embodiment, the first correction torque may be provided to the user for a preset time. For example, the first correction torque may be output by the wearable device for 20 minutes during which the user is walking.

The walking state of the user may be improved by the first correction torque including the first torque and the first additional torque. For example, by the first torque and the first additional torque, the pelvic movement range of the user may be reduced, the range between the maximum front angle and the maximum rear angle of the leg of the user may be increased, or the angle range between the first partial leg support frame 820 and the second partial leg support frame 830 may be reduced.

FIG. 10 is a diagram illustrating a method of obtaining straight leg movement information of a user according to an example embodiment.

According to an example embodiment, the wearable device 100 (e.g., the wearable device 800 of FIG. 8) described above with reference to FIG. 1 may measure (or sense) a left hip joint angle q_l and a right hip joint angle q_r of a user. For example, the wearable device 100 may measure the right hip joint angle q_r of the user through a right angle sensor (e.g., the first angle sensor 524 of FIG. 5B) and the left hip joint angle q_l of the user through a left angle sensor (e.g., the second angle sensor 524-1 of FIG. 5B). As shown in FIG. 10, the left hip joint angle q_l may be a negative number because a left leg of the user is before a reference line 1010, and the right hip joint angle q_r may be a positive number because a right leg of the user is behind the reference line 1010. According to implementation, the right hip joint angle q_r may be a negative number when the right leg is before the reference line 1010, and the left hip joint angle q_l may be a positive number when the left leg is behind the reference line 1010. Based on the right hip joint angle q_r, maximum front angle information and maximum rear angle information of the right leg may be obtained. Based on the left hip joint angle q_l, maximum front angle information and maximum rear angle information of the left leg may be obtained.

According to an example embodiment, in walking for which a first correction torque is provided to the user, first correction torque information may be determined to increase a range between a maximum front angle and a maximum rear angle of the left/right leg of the user, compared to those in test walking.

According to an example embodiment, the wearable device 100 may obtain a first angle (e.g., q_r) and a second angle (e.g., q_l) by filtering a first raw angle (e.g., q_r_raw) of a first joint (e.g., the right hip joint) measured by the first angle sensor 524 and a second raw angle (e.g., q_l_raw) of a second joint (e.g., the left hip joint) measured by the second angle sensor 524-1. For example, the wearable device 100 may filter the first raw angle and the second raw angle based on a first previous angle and a second previous angle that are measured at a previous time.

According to an example embodiment, the wearable device 100 may determine a torque value τ(t) based on the left hip joint angle q_l, the right hip joint angle q_r, an offset angle c, a sensitivity α, a gain κ, and a delay Δt, and may control a motor driver circuit (e.g., the motor driver circuits 532 and 532-1) of the wearable device 100 to output the determined torque value τ(t). A force to be provided to the user by the torque value τ(t) may be referred to herein as force feedback. For example, the wearable device 100 may determine the torque value τ(t) based on Equation 1 below. The term “first torque value” may refer to the magnitude of a first torque output at a specific point in time.

$$y = sin ( q\_r ) - sin ( q\_l ) \qquad [ Equation 1 ]$$

$$τ ( t ) = κ y ( t - Δ t )$$

In Equation 1 above, y denotes a state factor, and q_r denotes a right hip joint angle and q_l denotes a left hip joint angle. According to Equation 1 above, the state factor y may be associated with a distance between both legs. For example, y being 0 may indicate a state (e.g., a crossing state) in which the distance between the legs is zero (0), and an absolute value of y being maximum may indicate a state (e.g., a landing state) in which an angle between the legs is maximum. According to an example embodiment, when q_r and q_l are measured at a time t, the state factor may be represented as y(t).

The gain may be a parameter indicating the magnitude and direction of an output torque. As the magnitude of the gain K increases, a greater torque may be output. When the gain k is a negative number, a torque acting as a resistance force may be output to the user. When the gain k is a positive number, a torque acting as an assistance force may be output to the user. The delay Δt may be a parameter associated with a torque output timing. A value of the gain x and a value of the delay Δt may be set in advance and may be adjusted by the user or the wearable device 100. Based on such parameters as Equation 1, the gain k, and the delay Δt, a model configured to output the torque acting as the assistance force to the user may be a torque output model (or a torque output algorithm). To the torque output model, values of input parameters received through sensors of the wearable device 100 may be input to determine the magnitude and delay of a torque to be output.

According to an example embodiment, the wearable device 100 may determine a first torque value through Equation 2 below by applying, to a first state factor y(t), a first gain value and a first delay value as parameter values determined for a state factor y(t).

$${τ}_{l} ( t ) = κ y ( t - Δ t ) \qquad [ Equation 2 ]$$

$${τ}_{r} ( t ) = - κ y ( t - Δ t )$$

It is required to be applied to both legs, and thus the calculated first torque value may include a value for the first joint and a value for the second joint. For example, τₗ(t) may be a value for the left hip joint which is the second joint, and τᵣ(t) may be a value for the right hip joint which is the first joint. τₗ(t) and τᵣ(t) may have the same magnitude but opposite torque directions. The wearable device 100 may control the motor driver circuits 532 and 532-1 of the wearable device 100 to output a torque corresponding to the first torque value. The first correction torque information may include the first torque described with reference to Equation 2 above.

According to an example embodiment, when the user walks with the left leg and the right leg being asymmetrical, the wearable device 100 may provide an asymmetric torque to both legs of the user to assist in such asymmetric walking. For example, a greater assistance force may be provided to a leg with a shorter stride or slower swing speed. Hereinafter, the leg with the shorter stride or slower swing speed will be referred to as an affected leg or target leg.

In general, the affected leg may have a shorter swing time or have a smaller stride than a sound leg or unaffected leg. According to an example embodiment, a method of adjusting a timing of a torque acting on the affected leg to assist the user in walking may be considered. For example, an offset angle may be added to an actual joint angle of the affected leg to increase an output time of a torque for assisting the affected leg with a swing motion. c may be a value of a parameter indicating an offset angle between joint angles. As the offset angle is added to the actual joint angle of the affected leg, a value of a parameter input to the torque output model provided in (or applied to) the wearable device 100 may be adjusted. For example, the values of q_r and q_l may be adjusted through Equation 3. cᵣ may denote an offset angle for the right hip joint and ci may denote an offset angle for the left hip joint.

$${q}_{\_ r} ( t ) ← {q}_{\_ r} ( t ) + {c}_{r} \qquad [ Equation 3 ]$$

$${q}_{\_ l} ( t ) ← {q}_{\_ l} ( t ) + {c}_{l}$$

According to an example embodiment, the wearable device 100 may filter a state factor to reduce discomfort the user may feel by an irregular torque output. For example, the wearable device 100 may determine an initial state factor y_{raw}(t) of a current time t based on a first angle of the first joint and a second angle of the second joint, and determine a first state factor y(t) based on a previous state factor y^{prv} determined for a previous time t−1 and the initial state factor y_{raw}(t). The current time t may indicate a processing time for tth data (or sample), and the previous time t−1 may indicate a processing time for t−1th data. For example, a difference between the current time t and the previous time t−1 may be an operation cycle of a processor that generates or processes the corresponding data. Sensitivity a may be a value of a parameter indicating sensitivity. For example, a sensitivity value may be continuously adjusted during test walking but be preset to a constant value to reduce computational complexity.

Although the method of determining values of control parameters by the wearable device 100 has been described above, the values of the control parameters may be determined by an electronic device (e.g., the electronic device 210 or the server 230 of FIG. 2), instead of the wearable device 100. For example, the electronic device may receive sensor data from the wearable device 100, determine the values of the control parameters based on the sensor data, and control operations of the wearable device 100 based on the determined values of the control parameters.

FIG. 11 is a diagram illustrating a method of obtaining test pelvic movement information of a user according to an example embodiment.

According to an example embodiment, when a user wears the wearable device 100 (e.g., the wearable device 800 of FIG. 8) described above with reference to FIG. 1, the IMU 135 of the wearable device 100 may be arranged to be positioned on a pelvis 1102 of the user. The IMU 135 may sense X-axis, Y-axis, and Z-axis angle ranges of a pelvic movement according to a movement of the user. As shown in FIG. 11, a side direction of the user may be set to an X-axis, a gravity direction may be set to a Y-axis, and a front direction of the user may be set to a Z-axis.

Through test walking of the user, test pelvic movement information of the user may be obtained. For example, the test pelvic movement information may include an X-axis angle range 1110, a Y-axis angle range 1120, and a Z-axis angle range 1130.

According to an example embodiment, the wearable device 100 may determine whether a walking state of the user is a normal state based on the test pelvic movement information and reference pelvic movement information. The reference pelvic movement information, which is information about a pelvic movement of a person who performs normal walking, may include an X-axis reference angle range, a Y-axis reference angle range, and a Z-axis reference angle range. For example, the X-axis reference angle range may be ±5°, the Y-axis reference angle range may be ±7°, and the Z-axis reference angle range may be ±4°. When values of the test pelvic movement information are within corresponding values of the reference pelvic movement information, the walking state of the user may be determined to be normal.

FIG. 12 is a diagram illustrating a method of obtaining lateral leg movement information of a user according to an example embodiment.

According to an example embodiment, an additional angle sensor included in a leg support frame (e.g., the leg support frame 810) of a wearable device (e.g., the wearable device 100 of FIG. 1 or the wearable device 800 of FIG. 8) may sense an angle between a first partial leg support frame (e.g., the first partial leg support frame 820) and a second partial leg support frame (e.g., the second partial leg support frame 830). For example, the additional angle sensor may directly sense an angle of the hinge 840. For example, the additional angle sensor, which is a sensor configured to measure a rotation angle of a motor of the additional drive module 850, may indirectly sense the angle between the first partial leg support frame 820 and the second partial leg support frame 830 based on the rotation angle of the motor of the additional drive module 850. For example, the additional angle sensor, which is a sensor configured to measure a position of the rod 860 of the additional drive module 850, may indirectly determine the angle between the first partial leg support frame 820 and the second partial leg support frame 830 based on the position of the rod 860.

For example, when a walking state of a user is not normal, the second partial leg support frame 830 may move in a lateral direction 1230 while the user is walking. In this case, through test walking, test lateral leg movement information of the user may be obtained.

According to an example embodiment, the wearable device may determine whether the walking state of the user is normal based on the test lateral leg movement information and reference lateral leg movement information. The reference lateral leg movement information may be information about a lateral leg movement of a person who performs normal walking, and values of the reference lateral leg movement information may be set in advance. When values of the test lateral leg movement information are within the values of the reference lateral leg movement information, the walking state of the user may be determined to be normal.

FIG. 13 is a flowchart illustrating a method of determining whether a walking state of a user is a normal state based on a test movement range and a reference movement range according to an example embodiment.

According to an example embodiment, operation 930 described above with reference to FIG. 9 may include operations 1310 through 1330 described below which may be performed by a wearable device (e.g., the wearable device 100 of FIG. 1 or the wearable device 800 of FIG. 8).

In operation 1310, the wearable device may calculate a difference between a test movement range and a reference movement range.

For example, in a case in which test movement information includes test pelvic movement information of a user obtained through an IMU (e.g., the IMU 135 of FIG. 1), a difference (hereinafter, a first difference) between values of the test pelvic movement information and values of the reference pelvic movement information may be calculated.

For example, in a case in which the test movement information includes straight leg movement information of the user obtained through an angle sensor, a difference (hereinafter, a second difference) between a value of test maximum front angle information and a value of test maximum rear angle information of a leg of the user and a value of reference maximum front angle information and a value of reference maximum rear angle information, respectively, may be calculated.

For example, in a case in which the test movement information includes angle information between a first partial leg support frame and a second partial leg support frame of a leg support frame, a difference (hereinafter, a third difference) between a value of the angle information between the first partial leg support frame and the second partial leg support frame and a value of reference angle information may be calculated.

In operation 1315, the wearable device may determine whether the difference between the test movement range and the reference movement range exceeds a preset first threshold value. For example, in a case in which the calculated difference between the test movement range and the reference movement range includes the first difference, the second difference, and the third difference, the first difference, the second difference, and the third difference may be compared to corresponding threshold values that are set in advance respectively for the first difference, the second difference, and the third difference.

In operation 1320, when the difference between the test movement range and the reference movement range exceeds the preset first threshold value, the wearable device may determine that a walking state of the user is not a normal state.

For example, in the case in which the difference between the test movement range and the reference movement range includes the first difference, the second difference, and the third difference, when any one of the first difference, the second difference, and the third difference exceeds a respectively set threshold value, the wearable device may determine that the walking state of the user is not the normal state.

In operation 1330, when the difference between the test movement range and the reference movement range does not exceed the preset first threshold value, the wearable device may determine that the walking state of the user is the normal state.

According to an example embodiment, when the walking state of the user is determined to be normal, operation 1510, which will be described below with reference to FIG. 15, may be performed.

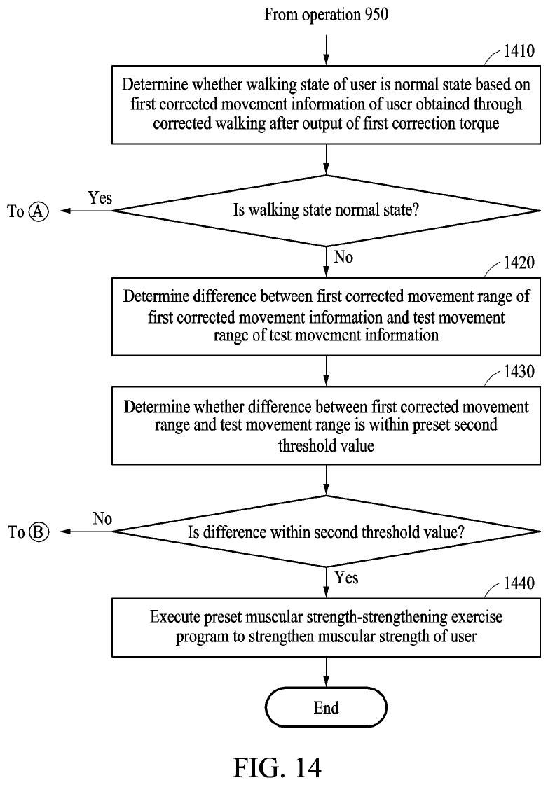

FIG. 14 is a flowchart illustrating a method of executing a muscular strength-strengthening exercise program based on a first corrected movement range and a test movement range according to an example embodiment.

According to an example embodiment, after operation 950 described above with reference to FIG. 9 is performed, operations 1410 to 1440 described below may be performed by a wearable device (e.g., the wearable device 100 of FIG. 1 or the wearable device 800 of FIG. 8).

In operation 1410, the wearable device may determine whether a walking state of a user is a normal state based on first corrected movement information of the user obtained through corrected walking after an output of a first correction torque. In this case, the wearable device may output, to the user, the first correction torque while the user is walking for corrected walking, unlike test walking.

For a method of obtaining the first corrected movement information, what has been described above regarding a method of obtaining test movement information may be applied in a similar way, and a more detailed and repeated description thereof is omitted here.

For a method of determining whether the walking state of the user is the normal state based on the first corrected movement information, what has been described above regarding operation 930 with reference to FIGS. 9 and 10 may be applied in a similar way, and a more detailed and repeated description thereof is omitted here.

According to an example embodiment, when the walking state of the user is determined to be normal, the following operation A may be performed to provide a muscular strength-assisting exercise program to the user. The operation A will be described in detail below with reference to FIGS. 15 and 16A.

According to an example embodiment, when the walking state of the user is not determined to be normal, operation 1420 may be performed.

In operation 1420, the wearable device may determine a difference between a first corrected movement range of the first corrected movement information and a test movement range of test movement information.

For a method of determining the difference between the first corrected movement range and the test movement range, what has been described above regarding a method of determining a difference between a test movement range and a reference movement range may be applied in a similar way. For example, the “test movement range” in operation 1310 described above with reference to FIG. 13 may be replaced here with the “first corrected movement range” and the “reference movement range” in operation 1310 described above with reference to FIG. 13 may be replaced here with the “test movement range.”

In operation 1430, the wearable device may determine whether the difference between the first corrected movement range and the test movement range is within a preset second threshold value.

For a method of determining whether the difference between the first corrected movement range and the test movement range is within the preset second threshold value, what has been described above regarding a method of determining whether a difference between a test movement range and a reference movement range exceeds a first threshold value may be applied in a similar way. For example, the “difference between the test movement range and the reference movement range” in operation 1315 described above with reference to FIG. 13 may be replaced here with the “difference between the first corrected movement range and the test movement range,” and the “first threshold value” in operation 1315 described above with reference to FIG. 13 may be replaced here with the “second threshold value.”

Operations 1420 and 1430 may be performed to check how much a movement of the user has been corrected by the first correction torque compared to a previous movement.

According to an example embodiment, when the difference between the first corrected movement range and the test movement range is not within the second threshold value, the following operation B may be performed. That the difference between the first corrected movement range and the test movement range is not within the second threshold value may indicate that walking of the user is being corrected by the first correction torque.

According to an example embodiment, when the difference between the first corrected movement range and the test movement range is within the second threshold value, operation 1440 may be performed. That the difference between the first corrected movement range and the test movement range is within the second threshold value may indicate that walking of the user is not being desirably corrected by the first correction torque.

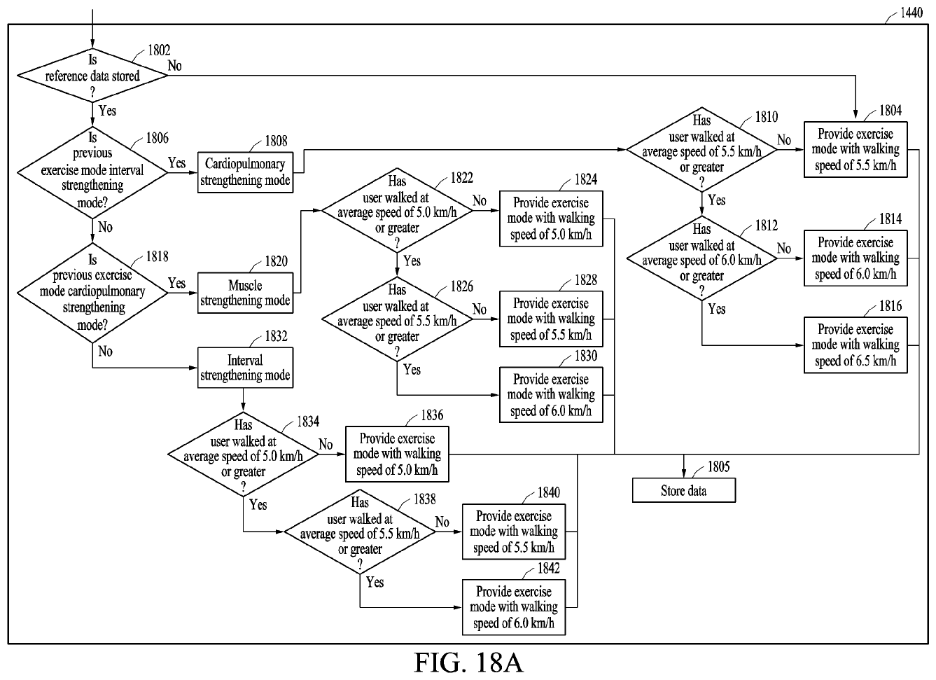

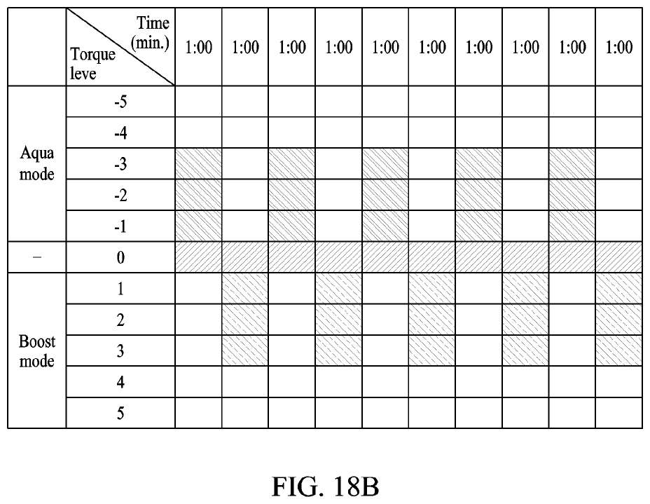

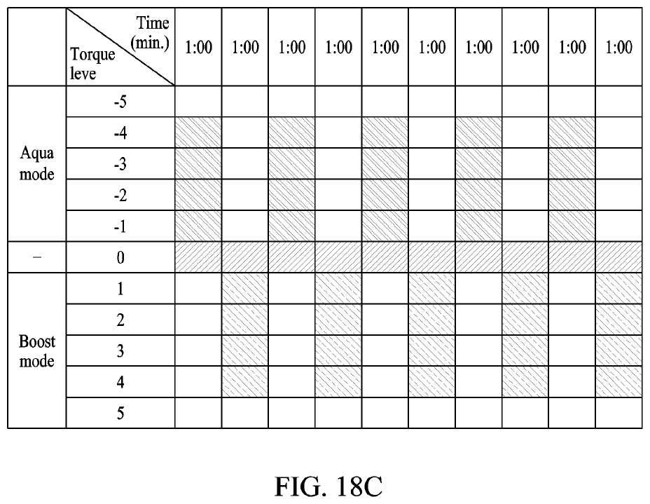

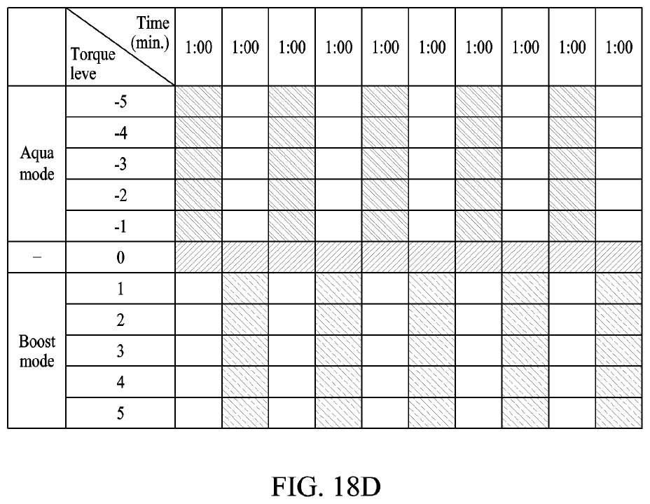

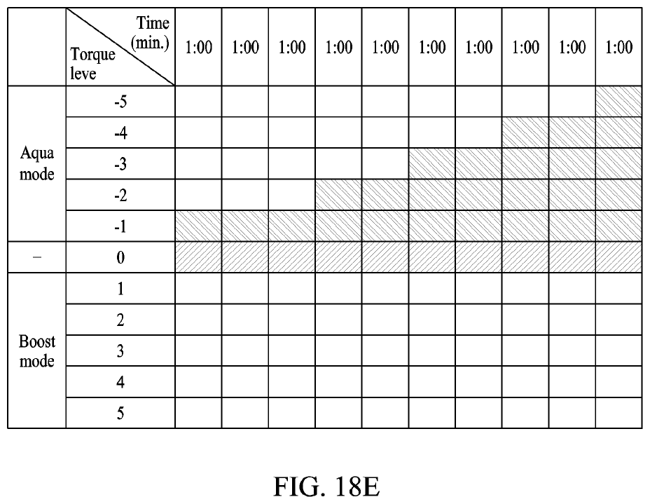

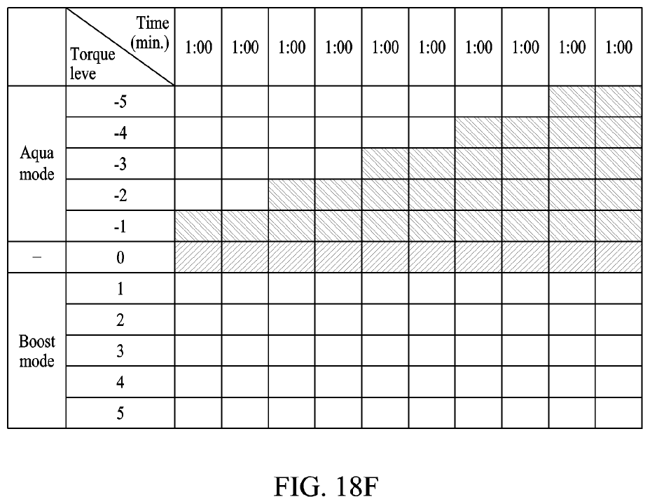

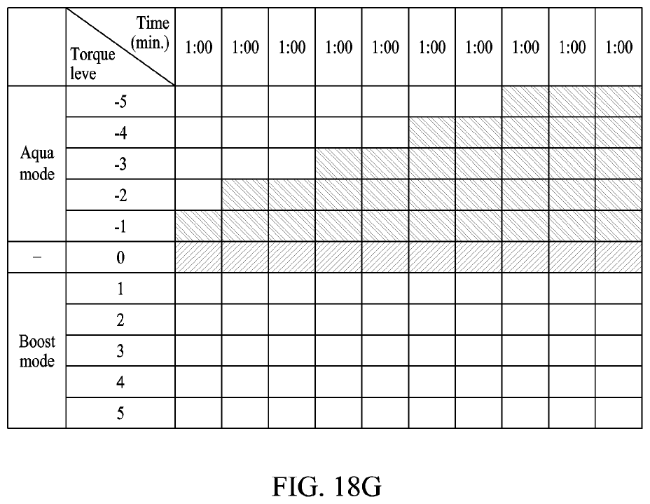

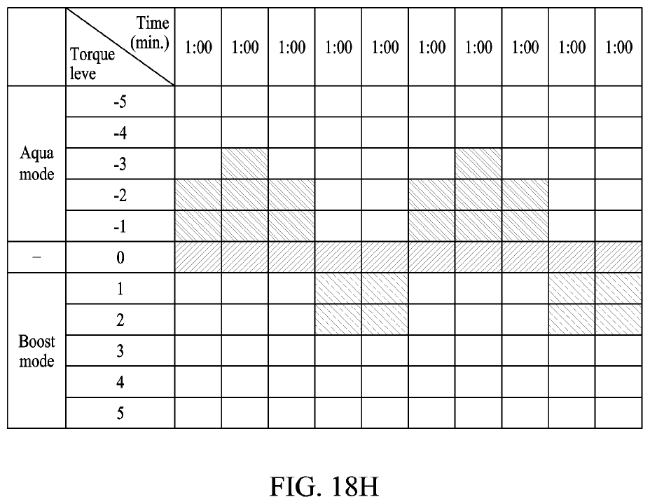

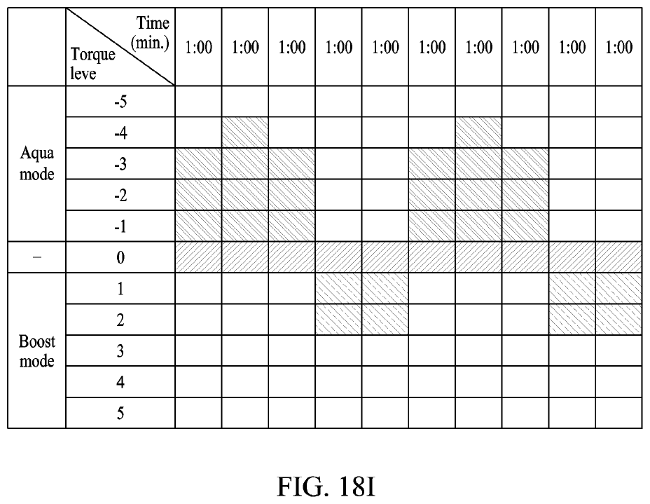

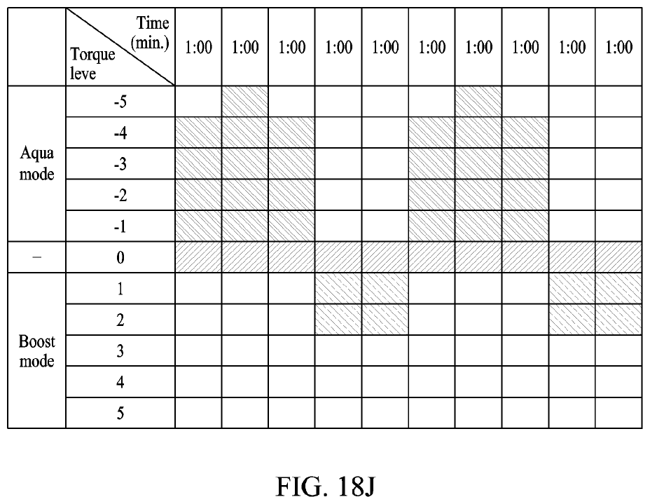

In operation 1440, when the difference between the first corrected movement range and the test movement range is within the second threshold value, the wearable device may execute a preset muscular strength-strengthening exercise program to strengthen muscular strength of the user. The muscular strength-strengthening exercise program will be described in detail below with reference to FIG. 18A.

FIG. 15 is a flowchart illustrating a method of executing a muscular strength-assisting exercise program in a case in which a walking state of a user is a normal state according to an example embodiment.

According to an example embodiment, when a walking state of a user is a normal state, the following operation 1510 may be performed. Operation 1510 may be performed by a wearable device (e.g., the wearable device 100 of FIG. 1 or the wearable device 800 of FIG. 8).

In operation 1510, when the walking state of the user is normal, the wearable device may execute a preset muscular strength-assisting exercise program to assist muscular strength of the user. Even when the walking state of the user is normal, an assistance torque may be provided to the user through the wearable device to increase a walking speed of the user. As the walking speed increases, oxygen intake of the user may increase, which may also increase calorie consumption.

According to an example embodiment, the muscular strength-assisting exercise program will be described in detail below with reference to FIG. 16A.

FIG. 16A is a flowchart illustrating a method of executing a muscular strength-assisting exercise program according to an example embodiment.

According to an example embodiment, operation 1510 described above with reference to FIG. 15 may include operations 1610 to 1670 described below which may be performed by a wearable device (e.g., the wearable device 100 of FIG. 1 or the wearable device 800 of FIG. 8).

In operation 1610, the wearable device may determine whether reference data of a user is stored. For example, the reference data may include the most recently measured movement information of the user. For example, the reference data may include a level of an exercise program performed by the user.

For example, when a level of a muscular strength-assisting exercise program performed by the user is stored as the reference data, operation 1620 may be performed. When the reference data is not stored, operation 1640 may be performed.

In operation 1620, the wearable device may determine whether the user has walked at an average speed of 4.5 kilometers per hour (km/h) or greater based on the reference data. When the user has walked at the average speed of 4.5 km/h or greater, operation 1630 may be performed. When the user has failed to walk at the average speed of 4.5 km/h or greater, operation 1640 may be performed.

In operation 1630, the wearable device may determine whether the user has walked at an average speed of 5.0 km/h or greater based on the reference data. When the user has walked at the average speed of 5.0 km/h or greater, operation 1660 may be performed. When the user has failed to walk at the average speed of 5.0 km/h or greater, operation 1650 may be performed.

In operation 1640, the wearable device may provide the user with an assistance mode that allows the user to achieve a goal of walking at a walking speed of 4.5 km/h. An example operation protocol of such a walking assistance mode provided to the user to allow the user to achieve the goal of walking at the walking speed of 4.5 km/h is described below with reference to Table 1.

TABLE 1

| | Operation protocol of walking assistance | | | | | | | | | |
|---|---|---|---|---|---|---|---|---|---|---|
| | mode for walking speed of 4.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | 2 | 3 | 4 | 3 | 2 |
| mode | | | | | | | | | | |
| Aqua | −1 | −2 | −3 | −2 | −1 | — | — | — | — | — |
| mode | | | | | | | | | | |
| Speed | 4.5 | 4.5 | 4.5 | 4.5 | 4.5 | 4.5 | 4.5 | 4.5 | 4.5 | 4.5 |
| (km/h) | | | | | | | | | | |

In Table 1, the boost mode may be a mode in which an assistance force is provided to assist the user in walking, and the aqua mode may be a mode in which a resistance force is provided to hinder the user from walking. For example, a positive torque value may be output in the boost mode, and a negative torque value may be output in the aqua mode. The values of the boost mode and the aqua mode presented in Table 1 may be a gain value used to calculate a torque value or a level representing the gain value. For example, levels −5, −4, −3, −2, −1, 1, 2, 3, 4, and 5 may respectively correspond to gains −9, −7.5, −6, −4, −2, 2, 4, 6, 7.5, and 9 Nm. The foregoing protocol may be provided to the user for a total of 10 minutes, and the magnitude of a torque provided to the user may change every minute. The operation protocol presented in Table 1 is shown in FIG. 16B.

In operation 1650, the wearable device may provide the user with an assistance mode that allows the user to achieve a goal of walking at a walking speed of 5.0 km/h. An example operation protocol of such a walking assistance mode provided to the user to allow the user to achieve the goal of walking at the walking speed of 5.0 km/h is described below with reference to Table 2.

TABLE 2

| | Operation protocol of walking assistance | | | | | | | | | |
|---|---|---|---|---|---|---|---|---|---|---|
| | mode for walking speed of 5.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | 2 | 3 | 4 | 3 | 2 |
| mode | | | | | | | | | | |
| Aqua | −1 | −2 | −3 | −2 | −1 | — | — | — | — | — |
| mode | | | | | | | | | | |
| Speed | 5.0 | 5.0 | 5.0 | 5.0 | 5.0 | 5.0 | 5.0 | 5.0 | 5.0 | 5.0 |
| (km/h) | | | | | | | | | | |

According to an example embodiment, a gain value of a torque of the operation protocol presented in Table 2 may be the same as the gain value of the operation protocol presented in Table 1, but as a walking speed of the user is set to 5.0 km/h, values of other parameters (e.g., delay) for outputting the torque may be different from the values of the corresponding parameters of the operation protocol presented in Table 1. The operation protocol presented in Table 2 may correspond to what is shown in FIG. 16B.

In operation 1660, the wearable device may provide the user with an assistance mode that allows the user to achieve a goal of walking at a walking speed of 5.5 km/h. An example operation protocol of such a walking assistance mode provided to the user to allow the user to achieve the goal of walking at the walking speed of 5.5 km/h is described below with reference to Table 3.

TABLE 3

| | Operation protocol of walking assistance | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | 2 | 3 | 4 | 3 | 2 |
| mode | | | | | | | | | | |
| Aqua | −1 | −2 | −3 | −2 | −1 | — | — | — | — | — |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

According to an example embodiment, a gain value of a torque of the operation protocol presented in Table 3 may be the same as the gain value of the operation protocol presented in Table 1 or Table 2, but as a walking speed of the user is set to 5.5 km/h, values of other parameters (e.g., delay) for outputting the torque may be different from the values of the corresponding parameters of the operation protocol presented in Table 1 or Table 2. The operation protocol presented in Table 3 may correspond to what is shown in FIG. 16B.

The assistance mode through operation 1640, operation 1650, or operation 1660 may provide the user with two or more cycles of one-time exercise in which a preset time (e.g., 10 minutes) is given for one cycle of the exercise. In this case, the user may be provided an additional time when desired.

In operation 1670, the wearable device may store data on an exercise performed by the user. For example, the wearable device may store movement information of the user measured during the exercise. For example, a level of the muscular strength-assisting exercise program performed by the user may be stored as the data.

According to an example embodiment, the wearable device may basically perform at least one cycle (e.g., two cycles) of the assistance mode. For example, the wearable device may basically perform two cycles of the assistance mode and then end performing the assistance mode. For example, after basically performing the two cycles of the assistance mode, the wearable device may ask the user whether to perform an additional cycle of the assistance mode and perform the additional cycle of the assistance mode based on a reply from the user.

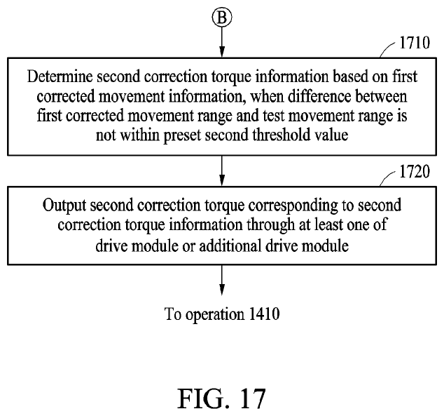

FIG. 17 is a flowchart illustrating a method of outputting a second correction torque for correcting a walking posture of a user according to an example embodiment.

According to an example embodiment, in operation 1430 described above with reference to FIG. 14, when it is determined that a difference between a first corrected movement range and a test movement range is not within a second threshold value, operations 1710 and 1720 below may be performed. Operations 1710 and 1720 may be performed by a wearable device (e.g., the wearable device 100 of FIG. 1 or the wearable device 800 of FIG. 8).

In operation 1710, the wearable device may determine second correction torque information based on first corrected movement information. For example, the second correction torque information may include a control signal for at least one of a drive module (e.g., the drive module 120 of FIG. 1) or an additional drive module (e.g., the additional drive module 850 of FIG. 8).

According to an example embodiment, the wearable device may determine the second correction torque information based on a difference between a first corrected movement range and a test movement range. For a method of determining the second correction torque information, what has been described above regarding operation 940 with reference to FIG. 9 may be applied in a similar way.

That the difference between the first corrected movement range and the test movement range is not within the second threshold value may indicate that the user is desirably adapted to a first correction torque. When the user is desirably adapted to the first correction torque, a correction torque greater than the first correction torque may be provided to the user. Although 5% is provided as an example of a preset ratio for calculating the first correction torque information in the foregoing description of operation 940, the ratio may be adjusted to exceed 5% to calculate the second correction torque information. The adjusted ratio may be determined to be proportional based on the difference between the first corrected movement range and the test movement range.

In operation 1720, the wearable device may output a second correction torque corresponding to the second correction torque information through at least one of the drive module (e.g., the drive module 120 of FIG. 1) or the additional drive module (e.g., the additional drive module 850 of FIG. 8). For example, the output second correction torque may be in the form of a torque trajectory corresponding to the entire walking cycle of the user. For example, when a left leg of the user steps forward, a second torque and a second additional torque may be output to assist the left leg in swinging, and when the left leg of the user steps backward, the second torque and the second additional torque may be output to assist the left leg in supporting.

FIG. 18A is a flowchart illustrating a method of executing a muscular strength-strengthening exercise program according to an example embodiment.

According to an example embodiment, operation 1440 described above with reference to FIG. 14 may include operations 1802 to 1842. Operations 1802 to 1842 may be performed by a wearable device (e.g., the wearable device 100 of FIG. 1 or the wearable device 800 of FIG. 8).

In operation 1802, the wearable device may determine whether reference data of a user is stored. For example, the reference data may include the most recently measured movement information of the user and may be, for example, a level of an exercise program performed by the user.

For example, when the level of the exercise program performed by the user is stored as the reference data, operation 1806 may be performed. When the reference data is not stored, operation 1804 may be performed.

In operation 1806, the wearable device may determine whether a previous exercise mode is an interval strengthening mode based on the reference data. When the previous exercise mode is the interval strengthening mode, operation 1808 may be performed. When the previous exercise mode is not the interval strengthening mode, operation 1818 may be performed.

In operation 1818, the wearable device may determine whether the previous exercise mode is a cardiopulmonary strengthening mode based on the reference data. When the previous exercise mode is the cardiopulmonary strengthening mode, operation 1820 may be performed. When the previous exercise mode is not the cardiopulmonary strengthening mode, operation 1832 may be performed.

In operation 1808, the wearable device may determine the exercise mode to be the cardiopulmonary strengthening mode.

In operation 1810, the wearable device may determine whether the user has walked at an average speed of 5.5 km/h or greater based on the reference data. When the user has walked at the average speed of 5.5 km/h or greater, operation 1812 may be performed. When the user has failed to walk at the average speed of 5.5 km/h or greater, operation 1804 may be performed.

In operation 1812, the wearable device may determine whether the user has walked at an average speed of 6.0 km/h or greater based on the reference data. When the user has walked at the average speed of 6.0 km/h or greater, operation 1816 may be performed. When the user has failed to walk at the average speed of 6.0 km/h or greater, operation 1814 may be performed.

In operation 1804, the wearable device may provide the user with an exercise mode that allows the user to walk at a walking speed of 5.5 km/h. Examples of an operation protocol of a cardiopulmonary strengthening mode provided to the user for strengthening a cardiopulmonary function while allowing the user to walk at the walking speed of 5.5 km/h will be described below with reference to Table 4, Table 5, and Table 6.

TABLE 4

| | Operation protocol of cardiopulmonary strengthening | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 3 | — | 3 | — | 3 | — | 3 | — | 3 |
| mode | | | | | | | | | | |
| Aqua | −3 | — | −3 | — | −3 | — | −3 | — | −3 | — |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

The operation protocol presented in Table 4 may combine an interval in which a strong resistance force is provided to the user and an interval in which a strong assistance force is provided to the user to dramatically change the heart rate of the user, which may, in turn, strengthen the cardiopulmonary function of the user. The operation protocol presented in Table 4 is shown in FIG. 18B.

TABLE 5

| | Operation protocol of cardiopulmonary strengthening | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 4 | — | 4 | — | 4 | — | 4 | — | 4 |
| mode | | | | | | | | | | |
| Aqua | −4 | — | −4 | — | −4 | — | −4 | — | −4 | — |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

The operation protocol presented in Table 5 may provide the user with a stronger resistance force and/or assistance force than the resistance force and/or assistance force in the operation protocol presented in Table 4. The operation protocol presented in Table 5 is shown in FIG. 18C.

TABLE 6

| | Operation protocol of cardiopulmonary strengthening | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 5 | — | 5 | — | 5 | — | 5 | — | 5 |
| mode | | | | | | | | | | |
| Aqua | −5 | — | −5 | — | −5 | — | −5 | — | −5 | — |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

The operation protocol presented in Table 6 may provide the user with a stronger resistance force and/or assistance force than the resistance force and/or assistance force in the operation protocol presented in Table 5. The operation protocol presented in Table 6 is shown in FIG. 18D.

In operation 1814, the wearable device may provide the user with an exercise mode that allows the user to walk at a walking speed of 6.0 km/h. Examples of an operation protocol of a cardiopulmonary strengthening mode provided to the user for strengthening a cardiopulmonary function while allowing the user to walk at the walking speed of 6.0 km/h will be described below with reference to Table 7, Table 8, and Table 9.

TABLE 7

| | Operation protocol of cardiopulmonary strengthening | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 3 | — | 3 | — | 3 | — | 3 | — | 3 |
| mode | | | | | | | | | | |
| Aqua | −3 | — | −3 | — | −3 | — | −3 | — | −3 | — |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

According to an example embodiment, a gain value of a torque in the operation protocol presented in Table 7 may be the same as a gain value in the operation protocol presented in Table 4, but as a walking speed of the user is set to 6.0 km/h, values of other parameters (e.g., delay) for outputting the torque may be different from the values of the corresponding parameters of the operation protocol presented in Table 4. The operation protocol presented in Table 7 may correspond to what is shown in FIG. 18B.

TABLE 8

| | Operation protocol of cardiopulmonary strengthening | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 4 | — | 4 | — | 4 | — | 4 | — | 4 |
| mode | | | | | | | | | | |
| Aqua | −4 | — | −4 | — | −4 | — | −4 | — | −4 | — |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

The operation protocol presented in Table 8 may provide the user with a stronger resistance force and/or assistance force than the resistance force and/or assistance force in the operation protocol presented in Table 7. The operation protocol presented in Table 8 is shown in FIG. 18C.

TABLE 9

| | Operation protocol of cardiopulmonary strengthening | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 5 | — | 5 | — | 5 | — | 5 | — | 5 |
| mode | | | | | | | | | | |
| Aqua | −5 | — | −5 | — | −5 | — | −5 | — | −5 | — |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

The operation protocol presented in Table 9 may provide the user with a stronger resistance force and/or assistance force than the resistance and/or assistance force in the operation protocol presented in Table 8. The operation protocol presented in Table 9 is shown in FIG. 18D.

In operation 1816, the wearable device may provide the user with an exercise mode that allows the user to walk at a walking speed of 6.5 km/h. Examples of an operation protocol of a cardiopulmonary strengthening mode provided to the user for strengthening a cardiopulmonary function while allowing the user to walk at the walking speed of 6.5 km/h will be described below with reference to Table 10, Table 11, and Table 12.

TABLE 10

| | Operation protocol of cardiopulmonary strengthening | | | | | | | | | |
|---|---|---|---|---|---|---|---|---|---|---|
| | mode for walking speed of 6.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 3 | — | 3 | — | 3 | — | 3 | — | 3 |
| mode | | | | | | | | | | |
| Aqua | −3 | — | −3 | — | −3 | — | −3 | — | −3 | — |
| mode | | | | | | | | | | |
| Speed | 6.5 | 6.5 | 6.5 | 6.5 | 6.5 | 6.5 | 6.5 | 6.5 | 6.5 | 6.5 |
| (km/h) | | | | | | | | | | |

According to an example embodiment, a gain value of a torque in the operation protocol presented in Table 10 may be the same as a gain value in the operation protocol presented in Table 4 or Table 7, but as a walking speed of the user is set to 6.5 km/h, values of other parameters (e.g., delay) for outputting the torque may be different from the values of the corresponding parameters of the operation protocol presented in Table 4 or Table 7. The operation protocol presented in Table 10 may correspond to what is shown in FIG. 18B.

TABLE 11

| | Operation protocol of cardiopulmonary strengthening | | | | | | | | | |
|---|---|---|---|---|---|---|---|---|---|---|
| | mode for walking speed of 6.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 4 | — | 4 | — | 4 | — | 4 | — | 4 |
| mode | | | | | | | | | | |
| Aqua | −4 | — | −4 | — | −4 | — | −4 | — | −4 | — |
| mode | | | | | | | | | | |
| Speed | 6.5 | 6.5 | 6.5 | 6.5 | 6.5 | 6.5 | 6.5 | 6.5 | 6.5 | 6.5 |
| (km/h) | | | | | | | | | | |

The operation protocol presented in Table 11 may provide the user with a stronger resistance force and/or assistance force than the resistance force and/or assistance force in the operation protocol presented in Table 10. The operation protocol presented in Table 11 may correspond to what is shown in FIG. 18C.

TABLE 12

| | Operation protocol of cardiopulmonary strengthening | | | | | | | | | |
|---|---|---|---|---|---|---|---|---|---|---|
| | mode for walking speed of 6.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | 5 | — | 5 | — | 5 | — | 5 | — | 5 |
| mode | | | | | | | | | | |
| Aqua | −5 | — | −5 | — | −5 | — | −5 | — | −5 | — |
| mode | | | | | | | | | | |
| Speed | 6.5 | 6.5 | 6.5 | 6.5 | 6.5 | 6.5 | 6.5 | 6.5 | 6.5 | 6.5 |
| (km/h) | | | | | | | | | | |

The operation protocol presented in Table 12 may provide the user with a stronger resistance force and/or assistance force than the resistance force and/or assistance force in the operation protocol presented in Table 11. The operation protocol presented in Table 12 may correspond to what is shown in FIG. 18D.

In operation 1820, the wearable device may determine the exercise mode to be a muscle strengthening mode.

In operation 1822, the wearable device may determine whether the user has walked at an average speed of 5.0 km/h or greater based on the reference data. When the user has walked at the average speed of 5.0 km/h or greater, operation 1826 may be performed. When the user has failed to walk at the average speed of 5.0 km/h or greater, operation 1824 may be performed.

In operation 1826, the wearable device may determine whether the user has walked at an average speed of 5.5 km/h or greater based on the reference data. When the user has walked at the average speed of 5.5 km/h or greater, operation 1830 may be performed. When the user has failed to walk at the average speed of 5.5 km/h or greater, operation 1828 may be performed.

In operation 1824, the wearable device may provide the user with an exercise mode that allows the user to walk at a walking speed of 5.0 km/h. Examples of an operation protocol of a muscle strengthening mode provided to the user for strengthening muscular strength of the user while allowing the user to walk at the walking speed of 5.0 km/h will be described below with reference to Table 13, Table 14, and Table 15.

TABLE 13

| | Operation protocol of muscle strengthening | | | | | | | | | |
|---|---|---|---|---|---|---|---|---|---|---|
| | mode for walking speed of 5.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −1 | −1 | −2 | −2 | −3 | −3 | −4 | −4 | −5 |
| mode | | | | | | | | | | |
| Speed | 5.0 | 5.0 | 5.0 | 5.0 | 5.0 | 5.0 | 5.0 | 5.0 | 5.0 | 5.0 |
| (km/h) | | | | | | | | | | |

The operation protocol presented in Table 13 may provide the user with a gradually increasing resistance force to strengthen the muscular strength of the user. The operation protocol presented in Table 4 may involve more adjacent muscles (e.g., hamstrings or glutei) in addition to main muscles (e.g., quadriceps femoris) in the front of a leg to promote muscle development through high muscle stimulation. The operation protocol presented in Table 13 is shown in FIG. 18E.

TABLE 14

| | Operation protocol of muscle strengthening | | | | | | | | | |
|---|---|---|---|---|---|---|---|---|---|---|
| | mode for walking speed of 5.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −1 | −2 | −2 | −3 | −3 | −4 | −4 | −5 | −5 |
| mode | | | | | | | | | | |
| Speed | 5.0 | 5.0 | 5.0 | 5.0 | 5.0 | 5.0 | 5.0 | 5.0 | 5.0 | 5.0 |
| km/h) | | | | | | | | | | |

The operation protocol presented in Table 14 may provide the user with a stronger resistance force than the resistance force in the operation protocol presented in Table 13. The operation protocol presented in Table 14 is shown in FIG. 18F.

TABLE 15

| | Operation protocol of muscle strengthening | | | | | | | | | |
|---|---|---|---|---|---|---|---|---|---|---|
| | mode for walking speed of 5.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −2 | −2 | −3 | −3 | −4 | −4 | −5 | −5 | −5 |
| mode | | | | | | | | | | |
| Speed | 5.0 | 5.0 | 5.0 | 5.0 | 5.0 | 5.0 | 5.0 | 5.0 | 5.0 | 5.0 |
| (km/h) | | | | | | | | | | |

The operation protocol presented in Table 15 may provide the user with a stronger resistance force than the resistance force in the operation protocol presented in Table 13 or Table 14. The operation protocol presented in Table 15 is shown in FIG. 18G.

In operation 1828, the wearable device may provide the user with an exercise mode that allows the user to walk at a walking speed of 5.5 km/h. Examples of an operation protocol of a muscle strengthening mode provided to the user for strengthening muscular strength of the user while allowing the user to walk at the walking speed of 5.0 km/h will be described below with reference to Table 16, Table 17, and Table 18.

TABLE 16

| | Operation protocol of muscle strengthening | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −1 | −1 | −2 | −2 | −3 | −3 | −4 | −4 | −5 |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

According to an example embodiment, a gain value of a torque in the operation protocol presented in Table 16 may be the same as a gain value in the operation protocol presented in Table 13, but as a walking speed of the user is set to 5.5 km/h, values of other parameters (e.g., delay) for outputting the torque may be different from the values of the corresponding parameters of the operation protocol presented in Table 13. The operation protocol presented in Table 16 may correspond to what is shown in FIG. 18E.

TABLE 17

| | Operation protocol of muscle strengthening | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −1 | −2 | −2 | −3 | −3 | −4 | −4 | −5 | −5 |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

The operation protocol presented in Table 17 may provide the user with a stronger resistance force than the resistance force in the operation protocol presented in Table 16. The operation protocol presented in Table 17 may correspond to what is shown in FIG. 18F.

TABLE 18

| | Operation protocol of muscle strengthening | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −2 | −2 | −3 | −3 | −4 | −4 | −5 | −5 | −5 |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

The operation protocol presented in Table 18 may provide the user with a stronger resistance force than the resistance force in the operation protocol presented in Table 17. The operation protocol presented in Table 18 may correspond to what is shown in FIG. 18G.

In operation 1830, the wearable device may provide the user with an exercise mode that allows the user to walk at a walking speed of 6.0 km/h. Examples of an operation protocol of a muscle strengthening mode provided to the user for strengthening muscular strength of the user while allowing the user to walk at the walking speed of 6.0 km/h will be described below with reference to Table 19, Table 20, and Table 21.

TABLE 19

| | Operation protocol of muscle strengthening | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −1 | −1 | −2 | −2 | −3 | −3 | −4 | −4 | −5 |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

According to an example embodiment, a gain value of a torque in the operation protocol presented in Table 19 may be the same as a gain value in the operation protocol presented in Table 16, but as a walking speed of the user is set to 6.0 km/h, values of other parameters (e.g., delay) for outputting the torque may be different from the values of the corresponding parameters of the operation protocol presented in Table 13 or Table 16. The operation protocol presented in Table 19 may correspond to what is shown in FIG. 18E.

TABLE 20

| | Operation protocol of muscle strengthening | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −1 | −2 | −2 | −3 | −3 | −4 | −4 | −5 | −5 |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

The operation protocol presented in Table 20 may provide the user with a stronger resistance force than the resistance force in the operation protocol presented in Table 19. The operation protocol presented in Table 20 may correspond to what is shown in FIG. 18F.

TABLE 21

| | Operation protocol of muscle strengthening | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | — | — | — | — | — | — | — |
| mode | | | | | | | | | | |
| Aqua | −1 | −2 | −2 | −3 | −3 | −4 | −4 | −5 | −5 | −5 |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

The operation protocol presented in Table 21 may provide the user with a stronger resistance force than the resistance force in the operation protocol presented in Table 20. The operation protocol presented in Table 21 may correspond to what is shown in FIG. 18G. In operation 1832, the wearable device may determine the exercise mode to be an interval strengthening mode.

In operation 1834, the wearable device may determine whether the user has walked at an average speed of 5.0 km/h or greater based on the reference data. When the user has walked at the average speed of 5.0 km/h or greater, operation 1838 may be performed. When the user has failed to walk at the average speed of 5.0 km/h or greater, operation 1836 may be performed.

In operation 1838, the wearable device may determine whether the user has walked at an average speed of 5.5 km/h or greater based on the reference data. When the user has walked at the average speed of 5.5 km/h or greater, operation 1842 may be performed. When the user has failed to walk at the average speed of 5.5 km/h or greater, operation 1840 may be performed.

In operation 1836, the wearable device may provide the user with an exercise mode that allows the user to walk at a walking speed of 5.0 km/h. Examples of an operation protocol of an interval training (or strengthening) mode provided to the user for training at intervals while allowing the user to walk at the walking speed of 5.0 km/h will be described below with reference to Table 22, Table 23, and Table 24.

TABLE 22

| | Operation protocol of interval training | | | | | | | | | |
|---|---|---|---|---|---|---|---|---|---|---|
| | mode for walking speed of 5.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −2 | −3 | −2 | — | — | −2 | −3 | −2 | — | — |
| mode | | | | | | | | | | |
| Speed | 5.0 | 5.0 | 5.0 | 5.0 | 5.0 | 5.0 | 5.0 | 5.0 | 5.0 | 5.0 |
| (km/h) | | | | | | | | | | |

The operation protocol presented in Table 22 may maximize an exercise effect in a sprint interval and assist the user in walking in a recovery interval to quickly stabilize the heart rate of the user, which may trigger a higher calorie consumption for the user compared to a general walking exercise. The operation protocol presented in Table 22 is shown in FIG. 18H.

TABLE 23

| | Operation protocol of interval training | | | | | | | | | |
|---|---|---|---|---|---|---|---|---|---|---|
| | mode for walking speed of 5.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −3 | −4 | −3 | — | — | −3 | −4 | −3 | — | — |
| mode | | | | | | | | | | |
| Speed | 5.0 | 5.0 | 5.0 | 5.0 | 5.0 | 5.0 | 5.0 | 5.0 | 5.0 | 5.0 |
| (km/h) | | | | | | | | | | |

The operation protocol presented in Table 23 may provide the user with a stronger resistance force than the resistance force in the operation protocol presented in Table 22. The operation protocol presented in Table 23 is shown in FIG. 18I.

TABLE 24

| | Operation protocol of interval training | | | | | | | | | |
|---|---|---|---|---|---|---|---|---|---|---|
| | mode for walking speed of 5.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −4 | −5 | −4 | — | — | −4 | −5 | −4 | — | — |
| mode | | | | | | | | | | |
| Speed | 5.0 | 5.0 | 5.0 | 5.0 | 5.0 | 5.0 | 5.0 | 5.0 | 5.0 | 5.0 |
| (km/h) | | | | | | | | | | |

The operation protocol presented in Table 24 may provide the user with a stronger resistance force than the resistance force in the operation protocol presented in Table 23. The operation protocol presented in Table 24 is shown in FIG. 18J.

In operation 1840, the wearable device may provide the user with an exercise mode that allows the user to walk at a walking speed of 5.5 km/h. Examples of an operation protocol of an interval training (or strengthening) mode provided to the user for training at intervals while allowing the user to walk at the walking speed of 5.5 km/h will be described below with reference to Table 25, Table 26, and Table 27.

TABLE 25

| | Operation protocol of interval training | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −2 | −3 | −2 | — | — | −2 | −3 | −2 | — | — |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

According to an example embodiment, a gain value of a torque in the operation protocol presented in Table 25 may be the same as a gain value in the operation protocol presented in Table 22, but as a walking speed of the user is set to 5.5 km/h, values of other parameters (e.g., delay) for outputting the torque may be different from the values of the corresponding parameters of the operation protocol presented in Table 22. The operation protocol presented in Table 25 may correspond to what is shown in FIG. 18H.

TABLE 26

| | Operation protocol of interval training | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −3 | −4 | −3 | — | — | −3 | −4 | −3 | — | — |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

The operation protocol presented in Table 26 may provide the user with a stronger resistance force than the resistance force in the operation protocol presented in Table 25. The operation protocol presented in Table 26 may correspond to what is shown in FIG. 18I.

TABLE 27

| | Operation protocol of interval training | | | | | | | | | |
|---|---|---|---|---|---|---|---|---|---|---|
| | mode for walking speed of 5.5 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −4 | −5 | −4 | — | — | −4 | −5 | −4 | — | — |
| mode | | | | | | | | | | |
| Speed | 5.5 | 5.5 | 5.5 | 5.5 | 5.5 | 5.5 | 5.5 | 5.5 | 5.5 | 5.5 |
| (km/h) | | | | | | | | | | |

The operation protocol presented in Table 27 may provide the user with a stronger resistance force than the resistance force in the operation protocol presented in Table 26. The operation protocol presented in Table 27 may correspond to what is shown in FIG. 18J.

In operation 1842, the wearable device may provide the user with an exercise mode that allows the user to walk at a walking speed of 6.0 km/h. Examples of an operation protocol of an interval training (or strengthening) mode provided to the user for training at intervals while allowing the user to walk at the walking speed of 6.0 km/h will be described below with reference to Table 28, Table 29, and Table 30.

TABLE 28

| | Operation protocol of interval training | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −2 | −3 | −2 | — | — | −2 | −3 | −2 | — | — |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

According to an example embodiment, a gain value of a torque in the operation protocol presented in Table 28 may be the same as a gain value in the operation protocol presented in Table 25, but as a walking speed of the user is set to 6.0 km/h, values of other parameters (e.g., delay) for outputting the torque may be different from the values of the corresponding parameters of the operation protocol presented in Table 27. The operation protocol presented in Table 28 may correspond to what is shown in FIG. 18H.

TABLE 29

| | Operation protocol of interval training | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −3 | −4 | −3 | — | — | −3 | −4 | −3 | — | — |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

The operation protocol presented in Table 29 may provide the user with a stronger resistance force than the resistance force in the operation protocol presented in Table 28. The operation protocol presented in Table 29 may correspond to what is shown in FIG. 18I.

TABLE 30

| | Operation protocol of interval training | | | | | | | | | |
|---|---|---|---|---|---|---|---|---|---|---|
| | mode for walking speed of 6.0 km/h | | | | | | | | | |
| Time | 1:00 | 1:00 | 1:00 | 1:00 | 1:00 | 1:00 | 1:00 | 1:00 | 1:00 | 1:00 |
| (min) | | | | | | | | | | |
| Boost | — | — | — | 2 | 2 | — | — | — | 2 | 2 |
| mode | | | | | | | | | | |
| Aqua | −4 | −5 | −4 | — | — | −4 | −5 | −4 | — | — |
| mode | | | | | | | | | | |
| Speed | 6.0 | 6.0 | 6.0 | 6.0 | 6.0 | 6.0 | 6.0 | 6.0 | 6.0 | 6.0 |
| (km/h) | | | | | | | | | | |

The operation protocol presented in Table 30 may provide the user with a stronger resistance force than the resistance force in the operation protocol presented in Table 29. The operation protocol presented in Table 30 may correspond to what is shown in FIG. 18J.

In operation 1805, the wearable device may store data on an exercise performed by the user. For example, movement information of the user measured during the exercise may be stored. For example, a level of an exercise program performed by the user may be stored as the data.

According to an example embodiment, the wearable device may basically perform at least one cycle (e.g., two cycles) of an exercise mode. For example, the wearable device may basically perform two cycles of the exercise mode and then end performing the exercise mode. For example, after basically performing the two cycles of the exercise mode, the wearable device may ask the user whether to perform an additional cycle of the exercise mode and perform the additional cycle of the exercise mode based on a reply from the user.

A method of improving a walking state of a user by providing a correction torque to the user, which is described above with reference to FIGS. 1 to 18A, may be provided on a one-time basis after the user wears a wearable device, and may also be provided to the user for a long period of time. For example, a muscular strength-strengthening exercise program using the wearable device may be provided to the user for several weeks (e.g., 4 to 8 weeks). Using such a long-term use of the wearable device, the user may improve their walking state or condition.

According to an example embodiment, a wearable device (e.g., 100 and 800) may include: a base body (e.g., 80) disposed proximate to a waist of a user (e.g., 110) when the wearable device is worn on a body of the user; a waist support frame (e.g., 20) and a leg support frame (e.g., 50, 55, and 810) configured to support at least a portion of the body of the user; a thigh fastener (e.g., 1 and 2) configured to attach the leg support frame to a thigh of the user; an IMU (e.g., 135) disposed within the base body; a drive module (e.g., 35, 45, and 120) configured to generate a torque to be applied to legs of the user, wherein the drive module is disposed between the waist support frame and the leg support frame; an angle sensor (e.g., 125) configured to measure a rotation angle of the leg support frame; and a control module (e.g., 130 and 510) including at least one processor configured to control the wearable device.

According to an example embodiment, the leg support frame may include: a first partial leg support frame (e.g., 820) connected, directly or indirectly, to the drive module; a second partial leg support frame (e.g., 830) connected, directly or indirectly, to the thigh fastener; a hinge (e.g., 840) connecting the first partial leg support frame and the second partial leg support frame; and an additional drive module (e.g., 850) configured to control a movement of the second partial leg support frame with respect to the first partial leg support frame.

According to an example embodiment, the wearable device may further include a battery configured to supply power to the wearable device.

According to an example embodiment, the wearable device may further include a communication module (e.g., 516) configured to perform short-range wireless communication with an external device (e.g., 210).

According to an example embodiment, the leg support frame may further include an additional angle sensor configured to measure an angle between the first partial leg support frame and the second partial leg support frame.

According to an example embodiment, the additional drive module may include a linear actuator.

According to an example embodiment, the at least one processor may perform: an operation (e.g., 930) of determining whether a walking state of the user is a normal state based on test movement information of the user obtained through test walking; an operation (e.g., 940) of determining first correction torque information based on the test movement information, when the walking state is not the normal state, wherein the first correction torque information may include a control signal for at least one of the drive module or the additional drive module; and an operation (e.g., 950) of outputting a first correction torque corresponding to the first correction torque information through at least one of the drive module or the additional drive module.

According to an example embodiment, the at least one processor may further perform: an operation (e.g., 910) of determining whether the wearable device is normally worn on the body of the user; and an operation (e.g., 920) of obtaining the test movement information when the wearable device is normally worn on the body of the user.

According to an example embodiment, the test movement information may include test pelvic movement information of the user obtained through the IMU.

According to an example embodiment, the test movement information may include straight leg movement information of the user obtained through the angle sensor.

According to an example embodiment, the test movement information may include lateral leg movement information of the user obtained through the additional angle sensor that measures an angle between the first partial leg support frame and the second partial leg support frame of the leg support frame.

According to an example embodiment, the operation (e.g., 930) of determining whether the walking state of the user is the normal state may include: an operation of determining whether the walking state of the user is the normal state by comparing a test movement range obtained based on the test movement information and a preset reference movement range.

According to an example embodiment, the operation of determining whether the walking state of the user is the normal state by comparing the test movement range obtained based on the test movement information and the preset reference movement range may include: an operation (e.g., 1310) of calculating a difference between the test movement range and the reference movement range; and an operation (e.g., 1320) of determining that the walking state of the user is not the normal state when the difference between the test movement range and the reference movement range exceeds a preset first threshold value.

According to an example embodiment, when the walking state is not the normal state, the operation (e.g., 940) of determining the first correction torque information based on the test movement information may include: an operation of determining the first correction torque information based on a difference between the test movement range and the preset reference movement range.

According to an example embodiment, the at least one processor may further perform: an operation (e.g., 1410) of determining whether the walking state of the user is the normal state based on first corrected movement information of the user obtained through corrected walking after an output of a first correction torque; an operation (e.g., 1420) of determining a difference between a first corrected movement range of the first corrected movement information and a test movement range of the test movement information, when the walking state is not the normal state; and an operation (e.g., 1440) of executing a preset muscular strength-strengthening exercise program to strengthen muscular strength of the user, when the difference between the first corrected movement range and the test movement range is within a preset second threshold value.

According to an example embodiment, when the walking state is the normal state, the at least one processor may further perform an operation (e.g., 1510) of executing a preset muscular strength-assisting exercise program to assist muscular strength of the user.

According to an example embodiment, a method of controlling a wearable device (e.g., 100 and 800) performed by the wearable device may include: an operation (e.g., 930) of determining whether a walking state of a user of the wearable device is a normal state based on test movement information of the user obtained through test walking; an operation (e.g., 940) of determining first correction torque information based on the test movement information, when the walking state is not the normal state, wherein the first correction torque information may include a control signal for at least one of a drive module or an additional drive module of the wearable device; and an operation (e.g., 950) of outputting a first correction torque corresponding to the first correction torque information through at least one of the drive module or the additional drive module. “Based on” as used herein covers based at least on.

According to an example embodiment, the method of controlling the wearable device may further include: an operation (e.g., 910) of determining whether the wearable device is normally worn on a body of the user; and an operation (e.g., 920) of obtaining the test movement information when the wearable device is normally worn on the body of the user.

According to an example embodiment, the operation (e.g., 930) of determining whether the walking state of the user is the normal state may include: an operation of determining whether the walking state is the normal state by comparing a test movement range obtained based on the test movement information and a preset reference movement range.

According to an example embodiment, the method of controlling the wearable device may further include: an operation (e.g., 1410) of determining whether the walking state of the user is the normal state based on first corrected movement information of the user obtained through corrected walking after an output of the first correction torque; an operation (e.g., 1420) of determining a difference between a first corrected movement range of the first correction movement information and a test movement range of the test movement information, when the walking state is not the normal state; and an operation (e.g., 1440) of executing a preset muscular strength-strengthening exercise program to strengthen muscular strength of the user, when the difference between the first corrected movement range and the test movement range is within a preset second threshold value.

Each embodiment herein may be used in combination with any other embodiment(s) described herein.

According to an example embodiment, the method of controlling the wearable device may further include an operation (e.g., 1510) of executing a preset muscular strength-assisting exercise program to assist the muscular strength of the user, when the walking state is the normal state.

The example embodiments described herein may be implemented using hardware components, software components and/or combinations thereof. A processing device may be implemented using one or more general-purpose or special purpose computers, such as, for example, at least one processor, a controller and an arithmetic logic unit (ALU), a digital signal processor, a microcomputer, a field programmable gate array (FPGA), a programmable logic unit (PLU), a microprocessor, or any other device capable of responding to and executing instructions in a given manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that a processing device may include multiple processing elements and multiple types of processing elements. For example, a processing device or processor may include multiple processors or a processor and a controller. In addition, different processing configurations are possible, such as parallel processors. Each “processor” herein comprises processing circuitry.

The software may include a computer program, a piece of code, an instruction, or some combination thereof, to independently or collectively instruct or configure the processing device to operate as desired. The software and/or data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be those specially designed and constructed for the purposes of examples, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described hardware devices may be configured to act as one or more software modules in order to perform the operations of the above-described examples, or vice versa. The term “software module” as used herein may include various processing circuitry and/or executable program instructions. The same applies to “software modules.” Each “module” herein may comprise circuitry.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. A wearable device, comprising:
a base body, comprising a housing, configured to be disposed proximate to a waist portion of a user;
a waist support frame and a leg support frame configured to support at least a portion of the body of the user;
a thigh fastener configured to attach the leg support frame to a thigh of the user;
an inertial measurement unit (IMU), comprising circuitry, disposed in the base body;
a drive module, comprising a motor and/or circuitry, configured to generate a torque to be applied to a leg of the user;
an angle sensor configured to measure a rotation angle of the leg support frame; and
a control module comprising at least one processor configured to control at least part of the wearable device,
wherein the leg support frame comprises:
a first partial leg support frame connected to the drive module;
a second partial leg support frame connected to the thigh fastener;
a hinge configured to connect the first partial leg support frame and the second partial leg support frame; and
an additional drive module, comprising an actuator and/or circuitry, configured to control a movement of the second partial leg support frame with respect to the first partial leg support frame.

2. The wearable device of claim 1, further comprising:
a battery configured to supply power to the wearable device.

3. The wearable device of claim 1, further comprising:
a communication module, comprising communication circuitry, configured to perform short-range wireless communication with an external device.

4. The wearable device of claim 1, wherein the leg support frame further comprises:
an additional angle sensor configured to measure an angle between the first partial leg support frame and the second partial leg support frame.

5. The wearable device of claim 1, wherein the additional drive module comprises a linear actuator.

6. The wearable device of claim 1, wherein the at least one processor is configured to perform:
an operation determining whether a walking state of the user is a normal state based on test movement information of the user obtained through test walking;
an operation determining first correction torque information based on the test movement information when the walking state is not the normal state, wherein the first correction torque information comprises a control signal for at least one of the drive module or the additional drive module; and
an operation outputting a first correction torque corresponding to the first correction torque information through at least one of the drive module or the additional drive module.

7. The wearable device of claim 6, wherein the at least one processor is configured to further perform:
an operation determining whether the wearable device is normally worn on the body of the user; and
an operation obtaining the test movement information when the wearable device is normally worn on the body of the user.

8. The wearable device of claim 7, wherein the test movement information comprises test pelvic movement information of the user obtained through the IMU.

9. The wearable device of claim 7, wherein the test movement information comprises straight leg movement information of the user obtained through the angle sensor.

10. The wearable device of claim 7, wherein the test movement information comprises lateral leg movement information of the user obtained through an additional angle sensor configured to measure an angle between the first partial leg support frame and the second partial leg support frame of the leg support frame.

11. The wearable device of claim 6, wherein the operation determining whether the walking state of the user is the normal state comprises:
determining whether the walking state of the user is the normal state at least by comparing a test movement range obtained based on the test movement information and a preset reference movement range.

12. The wearable device of claim 11, wherein the operation determining whether the walking state of the user is the normal state at least by comparing the test movement range obtained based on the test movement information and the preset reference movement range comprises:
calculating a difference between the test movement range and the reference movement range; and
determining that the walking state of the user is not the normal state, in response to the difference between the test movement range and the reference movement range exceeding a preset first threshold value.

13. The wearable device of claim 11, wherein the operation determining the first correction torque information based on the test movement information when the walking state is not the normal state comprises:
determining the first correction torque information based on a difference between the test movement range and the preset reference movement range.

14. The wearable device of claim 6, wherein the at least one processor is configured to further perform:
determining whether the walking state of the user is the normal state based on first corrected movement information of the user obtained through corrected walking after an output of the first correction torque;
determining a difference between a first corrected movement range of the first corrected movement information and a test movement range of the test movement information, when the walking state is not the normal state, and
executing a preset muscular strength-strengthening exercise program to strengthen a muscular strength of the user, in response to the difference between the first corrected movement range and the test movement range being within a preset second threshold value.

15. The wearable device of claim 6, wherein the at least one processor is configured to further perform:
executing a preset muscular strength-assisting exercise program to assist a muscular strength of the user, when the walking state is the normal state.

16. A method of controlling a wearable device performed by the wearable device, the method comprising:
determining whether a walking state of a user of the wearable device is a normal state based on test movement information of the user obtained through test walking;
determining first correction torque information based on the test movement information, when the walking state is not the normal state, wherein the first correction torque information comprises a control signal for at least one of a drive module or an additional drive module of the wearable device, each of the drive module and the additional drive module comprising a motor and/or circuitry; and
outputting a first correction torque corresponding to the first correction torque information through at least one of the drive module or the additional drive module.

17. The method of claim 16, further comprising:
determining whether the wearable device is normally worn on a body of the user; and
obtaining the test movement information when the wearable device is normally worn on the body of the user.

18. The method of claim 16, wherein the determining whether the walking state of the user is the normal state comprises:
determining whether the walking state of the user is the normal state at least by comparing a test movement range obtained based on the test movement information and a preset reference movement range.

19. The method of claim 16, further comprising:
determining whether the walking state of the user is the normal state based on first corrected movement information of the user obtained through corrected walking after an output of the first correction torque;
determining a difference between a first corrected movement range of the first corrected movement information and a test movement range of the test movement information, when the walking state is not the normal state; and
executing a preset muscular strength-strengthening exercise program to strengthen a muscular strength of the user, in response to the difference between the first corrected movement range and the test movement range being within a preset second threshold value.

20. The method of claim 16, further comprising:
executing a preset muscular strength-assisting exercise program to assist a muscular strength of the user, when the walking state is the normal state.
